(19) United States
(12) Patent Application Publication
(10) Pub. No.: US 2021/0046106 A1
(43) Pub. Date: Feb. 18, 2021

(54) METHODS OF TREATING CANCER WITH ANDROGEN DEPRIVATION THERAPY IN COMBINATION WITH AN INHIBITOR OF THE SP1 TRANSCRIPTION FACTOR

(71) Applicant: The Board of Trustees of the Leland Stanford Junior University, Stanford, CA (US)

(72) Inventors: Susan J. Knox, Stanford, CA (US); Donna M. Peehl, Mountain View, CA (US); Junqiang Tian, Salt Lake City, UT (US)

(21) Appl. No.: 16/883,744

(22) Filed: May 26, 2020

Related U.S. Application Data
(60) Provisional application No. 62/888,036, filed on Aug. 16, 2019.
(60) Provisional application No. 62/853,564, filed on May 28, 2019.

(51) Int. Cl. A61K 33/04; A61K 9/00; A61P 35/00; A61K 31/58; A61K 31/167; A61K 38/09; A61K 31/4166; A61N 5/10

(52) U.S. Cl. CPC A61K 33/04; A61K 9/0053; A61P 35/00; A61N 5/10; A61K 31/167; A61K 38/09; A61K 31/4166; A61K 31/58

(57) ABSTRACT

Methods for treating cancer using combination therapy with an inhibitor of the Sp1 transcription factor and radiation therapy are disclosed.

### CROSS REFERENCE TO RELATED APPLICATIONS

This application claims the benefit of U.S. Provisional Patent Application No. 62/888,036, filed Aug. 16, 2019, and U.S. Provisional Patent Application No. 62/853,564, filed May 28, 2019, which applications are incorporated herein by reference in its entireties.

### STATEMENT OF GOVERNMENT SUPPORT

This invention was made with Government support under contract W81XWH-04-1-0160 awarded by the Department of the Army. The Government has certain rights in this invention.

### BACKGROUND

Although cancer therapies have advanced greatly over the years, significant challenges remain. Cancer therapies are generally associated with undesirable side effects, highlighting the need for therapies that are selective for tumor cells, and thus have decreased toxicity. In addition, chemotherapy- and radiation-resistant cancers are very difficult to treat, with few therapeutic options, and low efficacy of available treatments. While there are multiple possible mechanisms of action of resistance to therapy, in some instances, this resistance is due to the resistance of the tumor cells to apoptosis and other forms of tumor cell killing.

For example, in prostate cancer, the resistance of prostate cancer cells to apoptosis plays a role in local and distant disease progression following conventional therapy (e.g., hormonal ablation and radiotherapy). The durable and local control rate (determined by serum levels of prostate specific antigen (PSA)) for patients with prostatic cancers of various stages and grades treated with primary radiation therapy alone is approximately 38%, and treatment of metastatic disease is palliative at best. The apoptotic machinery of most prostate cancer cells is intact, however, due to molecular alterations the cells may be unable to execute the apoptotic pathways.

Aberrant androgen receptor (AR) signaling plays a critical role in the development of castration-resistant prostate cancer (CRPC). Abiraterone, an inhibitor of androgen synthesis, is used to treat CRPC. Although abiraterone can suppress tumor growth in many patients with CRPC, most patients treated with abiraterone eventually become resistant to this drug. Recently, it has been reported that treatment with abiraterone increases the expression of AR splice variants 5, 6, and 7 (AR^{V567}s) in CRPC cells (Mostaghel et al. (2011) Clin. Cancer Res. 17(18):5913-5925). Several AR splice variants (AR-V7 and AR^{V567}s) have ligand-independent activity and are constitutively active.

Selenium, a key component of a number of functional selenoproteins required for normal health, when in the organic form, such as selenomethionine, or in the inorganic form, has been reported to have both preventive and therapeutic effects, respectively. Inorganic and organic selenite can inhibit tumorigenesis in a variety of animal models at doses in excess of those required to support maximal activity of selenoproteins (Ip, et al., Current concepts of selenium and mammary tumorigenesis, In: Cellular and Molecular Biology of Breast Cancer, 479-494. Plenum Press, N.Y. (1997); Medina et al., Pathol Immunopathol Res, 7: 187-199 (1988); Milner et al., Fed Proc, 44: 2568-2572 (1985)). Epidemiology studies have shown a statistically significant inverse relationship between selenium levels and cancer risk (Combs et al., Selenium and cancer, In: Antioxidants and Disease Prevention, Ch. 8, 97-113. CRC Press, N.Y. (1997); Shamberger et al., CRC Crit Rev Clin Sci, 2: 211-219 (1971)).

While the majority of selenium research has focused on the use of long-term selenium intake for chemoprevention, little attention has been given to the cytotoxic effects of selenium and the potential use of selenium, specifically inorganic selenium, as a systemic therapy in the clinical setting. The anti-tumor activities of selenium compounds are dependent upon the dose and chemical form. Selenite (oxidation state +4) undergoes thiol-dependent reduction to selenide (H₂Se), which supplies selenium for the synthesis of selenoproteins, whereas selenomethionine is converted to selenocysteine before being degraded by the enzyme β-lyase to H₂Se (Combs et al., Pharmacol. Ther., 79(3): 179-192 (1998)). Selenite metabolism results in the generation of superoxide radicals and oxidative stress as it also depletes glutathione (GSH) (FIG. 1) (Combs, 1998), the primary anti-oxidant in cells. Selenate is metabolized to selenite in the body.

Selenite is capable of inhibiting cell growth and inducing apoptosis in a variety of human cancer cells lines in vitro (Menter et al., Cancer Epid Bio Prey, 9: 1171-1182 (2000); Zhong et al., Cancer Res, 61: 7071-7078 (2001)). Selenite (2 mg/kg, subcutaneous injection) has also been shown to inhibit the tumor growth of breast and ovarian cancer cell lines in vivo without apparent ill effects on the host (Watrach et al., Cancer Letters, 25: 41-47 (1984); Watrach et al., Cancer Letters, 15: 137-143 (1982); Caffrey et al., Cancer Letters, 121: 177-180 (1997)). The induction of apoptosis by selenite is mediated by a redox mechanism involving induction of oxidative stress via superoxide formation and lowered intracellular GSH levels (Zhong, 2001). Mitochondria appear to serve as the main target for selenite-induced apoptosis, with subsequent release of cytochrome c, followed by mitochondrial depolarization, caspase-3 activation and DNA fragmentation (Shen et al., Free Rad Biol Med, 30(1): 9-21 (2001)). Several studies have also reported that selenium compounds selectively induce growth inhibition and apoptosis in cancer cells compared to normal cells (Menter, 2000; Fleming et al., Nut Cancer, 40(1): 42-49 (2001); Ghose et al., Cancer Res, 61: 7479-7487 (2001); Husbeck et al., Prostate, 66(2): 218-225 (2006)). However, the molecular pathways underlying the differential response are poorly understood. Selenite has also been shown to sensitize prostate cancer cells to y-irradiation. Both LAPC-4 and androgen-independent DU 145 cells pre-treated with selenite showed increased sensitivity to y-irradiation as measured by clonogenic survival assays (Husbeck, et al., Free Radic Biol Med, 38(1):50 7 2005)). Selenite-induced radiosensitization was also observed in vivo in LAPC-4 and PC3 tumors, but not in normal GI epithelium (Tian et al. Int. J. Radiat. Oncol. Biol. Phys. 78 (1): 230-236 (2010)). Furthermore, we demonstrated that inhibition of androgen receptor (AR) expression and activity by selenite occurs via a redox-mechanism involving GSH, superoxide and Sp1 (Husbeck et al, Mol Cancer Ther 5 (8): 2078-2085, 2006). Furthermore, treatment of mice with well-established prostate cancer tumors with selenite, resulted in downregulation of AR expression in vivo, and decreased levels of PSA (downstream of Sp1), that correlated with tumor growth inhibition (Bhattacharyya et al. Int J Radiat Oncol Biol Phys 72 (3): 935-940, 2008). These data suggest that altering the redox environment of prostate cancer cells with selenite increases the apoptotic potential, sensitizes them to radiation-induced cell killing, and downregulates AR expression and function via redox-mediated inhibition of Sp1.

There remains a need for improved methods of treating patients with cancers such as prostate cancer, particularly for patients who develop resistance to androgen deprivation therapy such as those undergoing treatment with abiraterone and enzalutamide.

### SUMMARY

Efficacious methods are provided for treating cancer. The methods utilize a combination therapy comprising administration of an inhibitor of the SP1 transcription factor, e.g., selenite, i.e. inorganic SeO₃²⁻, which may be provided as the sodium salt. Selenite is shown to act as a radiosensitizer, and can be administered in combination with radiation therapy, e.g. palliative radiation therapy, curative radiation therapy, and the like. In some embodiments, palliative radiation in combination with administration of selenite reduces cancer pain.

Cancer therapy may include treatment of hematologic cancers, e.g. lymphomas, leukemias, myelomas, etc. Cancer therapy may include treatment of solid cancers, e.g. lymphomas, carcinomas, sarcomas, gliomas, etc. For example, carcinomas may include squamous cell carcinoma, adenocarcinoma, adenosquamous carcinoma, anaplastic carcinoma, large cell carcinoma, neuroendocrine carcinoma, and small cell carcinoma.

In some embodiments the combination therapy is used to prevent or overcome resistance to drugs for androgen deprivation therapy. In some embodiments the cancer is androgen-responsive, androgen resistant, or androgen-independent cancer, including without limitation castrate resistant prostate cancer (CRPC). For the treatment of prostate cancer, selenite can be administered in combination with androgen deprivation therapy, including without limitation abiraterone, enzalutamide, etc.

Combined therapy of selenite with radiation therapy and optionally androgen deprivation therapy in CRPC can synergistically treat cancer; for example by decreasing growth of cancer cells. In a synergistic response, for example, the combination of agents can produce an effect that is greater than the effect of any of the agents administered as a monotherapy, and may be greater than the additive effect of each agent administered as a monotherapy. The addition of selenite may, for example, improve the level of treatment, such as reducing cancer growth, metastasis, etc., for a given dose of radiation and/or androgen deprivation therapy. The addition of selenite may allow the use of a lower dose of radiation and/or androgen deprivation therapy to achieve the same results. Therapy can be monitored, for example, by the level of PSA in the blood, by the growth of bone metastases, by decrease in pain, and the like as known in the art.

In some embodiments the agents in the combination are administered concomitantly, i.e. each agent is administered within about 45 days, 30 days, 15 days, 7 days, 3 days, 2 days, 1 day or substantially simultaneously with respect to the other agent(s) in the combination. The agents can be considered to be combined if administration scheduling is such that the serum level of both agents is at a therapeutic level. Administration may be repeated as necessary to treat the cancer.

In some embodiments, sodium selenite is delivered orally in combination with radiotherapy and/or androgen deprivation therapy. In some embodiments the dose of sodium selenite is from about 5 mg to about 60 mg delivered daily as a single dose, or up to about 100 mg in divided doses (e.g. 33 mg three times a day). Dosing may be up to about 11, about 16.5, about 33, about 45, about 50, up to about 60 mg daily, for example from 5 to 50 mg as a single oral dose. In some embodiments the target selenite concentration in the blood is about 5 to about 15 μM, e.g. from about 395 to about 790 mcg/L. In some embodiments the dose is administered as a single dose. In other embodiments the dose is fractioned into 2 doses; in some embodiments fractioned into 3 or more doses. Fractionated doses may be, for example, from about 5 to about 30 mg, from about 5 to about 25 mg, from about 5 to about 20 mg, from about 5 to about 15 mg, from about 5 to about 10 mg; usually administered once, twice, thrice daily. In certain embodiments the dose is administered from about 1 to 3 hours prior to radiotherapy.

In one aspect, a method of treating an androgen-responsive cancer or CRPC is provided comprising administering to a subject in need thereof a therapeutically effective amount of an inhibitor of a Sp1 transcription factor in combination with androgen deprivation therapy. Concurrent therapy can be performed by administering the inhibitor of the Sp1 transcription factor to a patient prior to, concurrent with, or subsequent to the androgen deprivation therapy as long as a combined therapeutic effect of the combination is achieved in the subject undergoing therapy. The treatment may be combined with radiotherapy.

In some embodiments the methods described herein are used to treat an androgen-responsive cancer, including, but not limited to, prostate cancer, breast cancer (e.g., triple-negative breast cancer), salivary gland cancer (e.g., salivary duct carcinoma), bladder cancer, and esophageal cancer (e.g., esophageal adenocarcinoma).

In certain embodiments, the inhibitor of the Sp1 transcription factor is selenite or a pharmaceutically acceptable salt thereof (e.g., a sodium or potassium salt).

In other embodiments, other inhibitors of the Sp1 transcription factor are used in treatment, such as, but not limited to, Withaferin A, mithramycin (aureolic acid, plicamycin) and analogues such as mithramycin SDK (SDK), mithramycin SK (SK), and premithrmycin B (PreB), 17-allylamino-17-demethoxygeldanamycin (17-AAG), EC-8042, bisanthracycline WP631, tolfenamic acid (clotam), tripartite motif 22 (TRIM22) peptide inhibitors, anti-sense, small interfering RNA (siRNA), or other nucleic acid inhibitors; and derivatives and analogues thereof.

The therapeutic agents used in combination therapy (i.e., the inhibitor of the Sp1 transcription factor and any therapeutic agent used in androgen deprivation therapy) may be administered by any suitable mode of administration. For example, therapeutic agents may be administered orally, intravenously, intramuscularly, or subcutaneously. Alternatively, the therapeutic agents may be administered locally at the site of a tumor. The inhibitor of the Sp1 transcription factor and any therapeutic agent used in androgen deprivation therapy may be administered by the same route or different routes.

In certain embodiments, the androgen deprivation therapy comprises administering a therapeutically effective amount of a luteinizing hormone-releasing hormone (LHRH) agonist or antagonist, a CYP17 inhibitor, an anti-androgen, or other androgen-suppressing agent. Exemplary LHRH agonists and antagonists include leuprolide, goserelin, triptorelin, histrelin, buserelin, and degarelix. Exemplary CYP17 inhibitors include abiraterone, ketoconazole, orteronel, galeterone, and seviteronel. Exemplary anti-androgens include cyproterone acetate, enzalutamide, apalutamide, flutamide, bicalutamide, and nilutamide. Other androgen-suppressing agents may include estrogen and derivatives and analogues thereof.

In certain embodiments, the androgen-responsive cancer is prostate cancer. In some embodiments, the prostate cancer is CRPC.

In certain embodiments, multiple cycles of treatment are administered to the subject for a time period sufficient to effect at least stabilization of disease, and preferably a partial tumor response, or more preferably, a complete tumor response. Treatment may be continued until cancer recurrence or progression indicates a change in therapy is required. In certain embodiments, the time period is at least 3 months. In other embodiments, the time period is at least 12 months. In certain embodiments, one or more cycles of treatment with the inhibitor of the Sp1 transcription factor is administered in combination with the androgen deprivation therapy during a treatment period, wherein said treatment period comprises a first time period wherein the inhibitor of the Sp1 transcription factor is administered to said subject with or without a rest period wherein no inhibitor of the Sp1 transcription factor is administered to the subject.

In some embodiments, the Sp1 transcription factor or the androgen deprivation therapy therapeutic agent is administered according to a daily dosing regimen or intermittently. The inhibitor of the Sp1 transcription factor and the androgen deprivation therapy therapeutic agent may be administered according to different dosing regimens. For example, the inhibitor of the Sp1 transcription factor may be administered according to a daily dosing regimen (e.g., one or multiple doses per day) or other schedule, and the androgen deprivation therapy therapeutic agent may be administered one day a week, two days a week, three days a week, four days a week, five days a week, six days a week, or seven days a week, and/or according to the standard of care schedule.

The methods disclosed herein may be combined with other anti-cancer treatment regimens, including one or more other anti-cancer therapies for treating cancer such as surgery, chemotherapy, radiation therapy, immunotherapy/biological therapy, hormonal therapy, cryotherapy, high-intensity focused ultrasound (HIFU), and photodynamic therapy.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIGS. 1A-1D show the effect of selenite on LAPC-4 cell proliferation and AR and PSA expression. FIG. 1A, LAPC-4 cells were treated with selenite at the indicated concentrations for 24, 48, and 72 h and cell proliferation was measured by MTT assay. FIG. 1B, LAPC-4 cells were cotransfected with the Androgen Receptor (AR) promoter-luciferase construct, pAR-luc, and pSV40-ren and then treated with selenite for 24 h. Luciferase activity was normalized to Renilla and expressed as percentage of control. FIG. 1C, AR protein expression in LAPC-4 cells after exposure to selenite for 24 h as detected by Western blot analysis. Actin protein expression was used to normalize for loading. FIG. 1D, LAPC-4 cells were treated with 10 μmol/L selenite for 6, 12, and 24 h and AR and PSA mRNA was measured by real-time reverse transcription-polymerase chain reaction (PCR). The expression of TATA box-binding protein was used for normalization. Points, mean of three experiments; bars, SD.

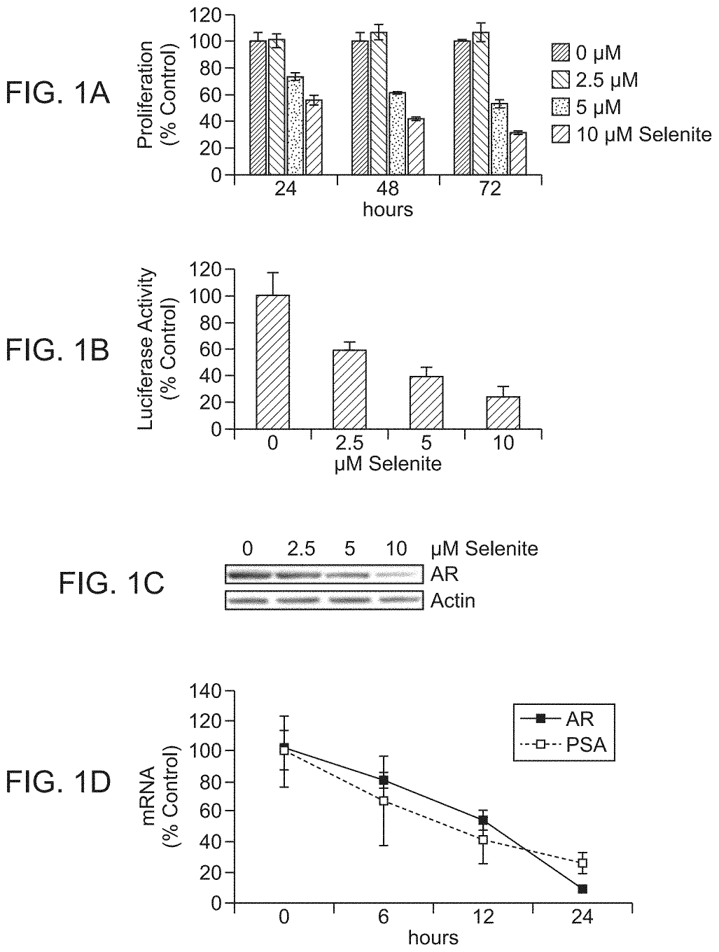

FIGS. 2A and 2B show that selenite inhibits the synthetic androgen, R1881-induced PSA expression. FIG. 2A, increasing amounts of R1881 were added to LAPC-4 cells growing in hormone-depleted medium and cellular PSA was detected by Western blot analysis 24 h later. Actin protein expression was used to normalize for loading. FIG. 2B, Enzyme-linked immunosorbent assay (ELISA) detection of secreted PSA in the conditioned medium from the same cells. PSA values were normalized to total protein per sample. Columns, mean of three experiments; bars, SD.

FIGS. 3A-3D show that N-acetylcysteine (NAC) inhibits selenite-induced down-regulation of the AR and PSA. LAPC-4 cells were pretreated with 10 mmol/L NAC for 24 h and then treated with selenite for another 24 h. FIG. 3A, AR protein expression determined by Western blot analysis after exposure to 5 or 10 μmol/L. Actin protein expression was used to normalize for loading. FIG. 3B, functional AR levels measured by [³H]-dihydrotestosterone (³H-DHT) binding. FIG. 3C, AR and PSA mRNA measured by real-time reverse transcription-PCR. FIG. 3D, ELISA detection of secreted PSA after exposure to 10 μmol/L selenite with or without NAC pretreatment. Columns, mean of three experiments; bars, SD.

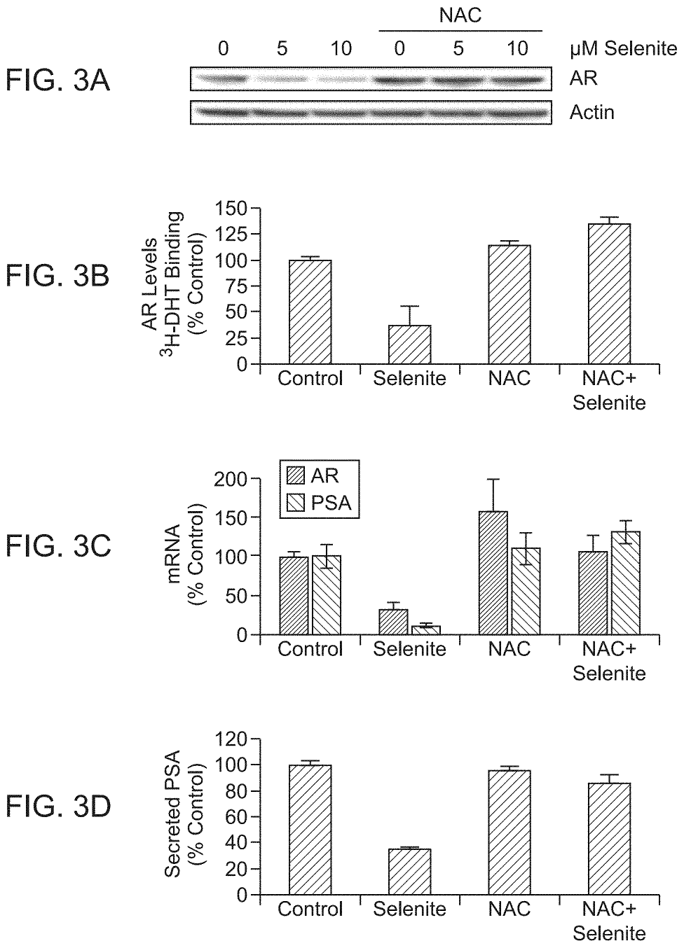

FIGS. 4A-4E show that NAC does not inhibit methylseleninic acid (MSeA) -induced down-regulation of the AR and PSA. FIG. 4A, LAPC-4 cells were pretreated with 10 mmol/L NAC for 24 h and then treated with 10 μmol/L MSeA for another 24 h and AR protein expression was detected by Western blot analysis. FIG. 4B, ELISA detection of secreted PSA from the same cells. LNCaP cells were pretreated with 10 mmol/L NAC for 24 h and then treated with 5 μmol/L selenite (FIG. 4C) or MSeA (FIG. 4D) for 24 h and AR protein expression was detected by Western blot analysis. FIG. 4E, ELISA detection of secreted PSA from the same cells. Actin protein expression was used to normalize for loading. Columns, mean of three experiments; bars, SD.

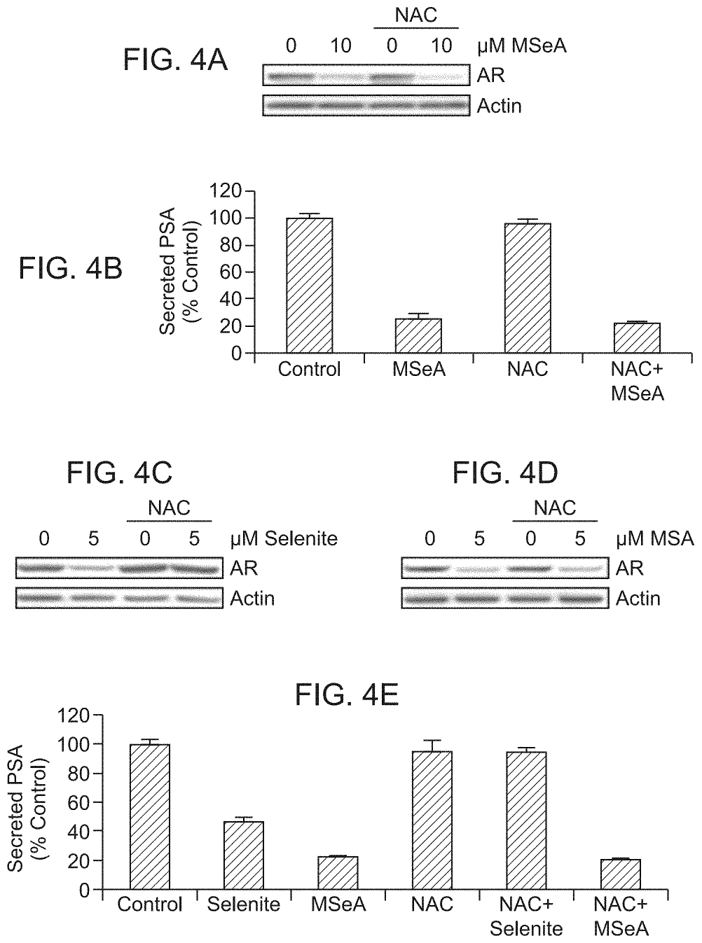

FIGS. 5A and 5B show the role of superoxide in selenite- and MSeA-induced inhibition of AR expression. LAPC-4 and LNCaP cells were treated with selenite or MSeA in the presence or absence of 5 μmol/L MnTMPyP (superoxide dismutase mimetic). FIG. 5A, Western blot analysis of AR protein expression in LAPC-4 cells 24 h after treatment with 10 μmol/L selenite or MSeA. FIG. 5B, LNCaP cells 24 h after treatment with 5 μmol/L selenite or MSeA. Actin protein expression was used to normalize for loading.

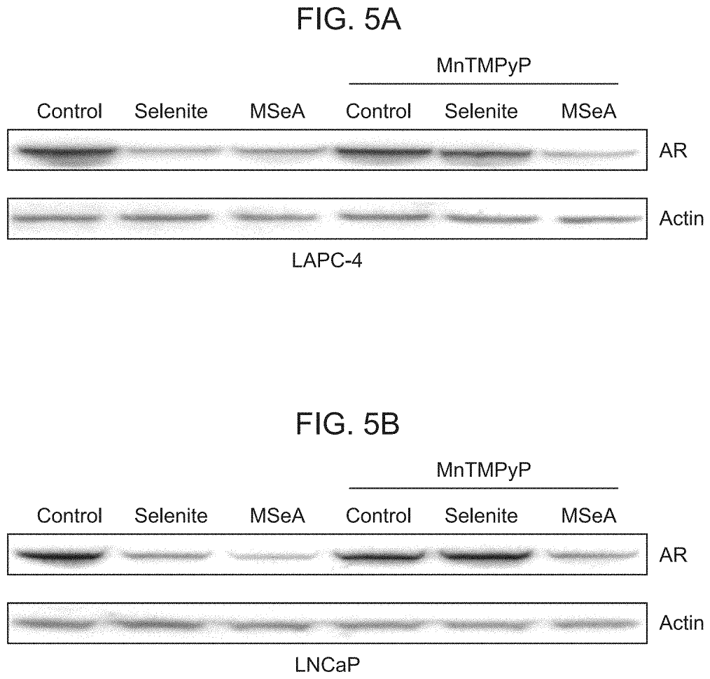

FIGS. 6A and 6B show the effects of selenite and MSeA on Sp1. FIG. 6A, LAPC-4 cells were cotransfected with the Sp1 reporter vector, pSp1-luciferase, and pSV40-renilla and then treated with 10 μmol/L selenite for 8 h. Luciferase activity was normalized to Renilla and expressed as percentage of control. Columns, mean of three experiments; bars, SD. FIG. 6B, Western blot analysis of Sp1 protein expression in the nuclear extracts of LAPC-4 and LNCaP cells exposed to selenite or MSeA for 8 h with or without NAC pretreatment. Ponceau S-stained bands were used to show equal loading of samples.

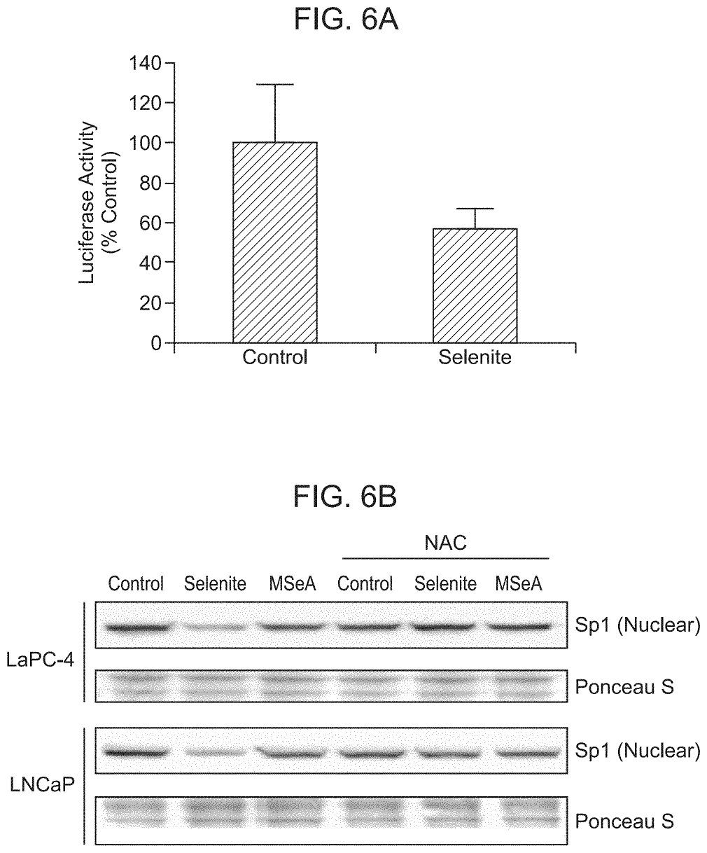

FIG. 7 shows a schematic illustration showing the inhibition of AR expression by selenite and MSeA in prostate cancer.

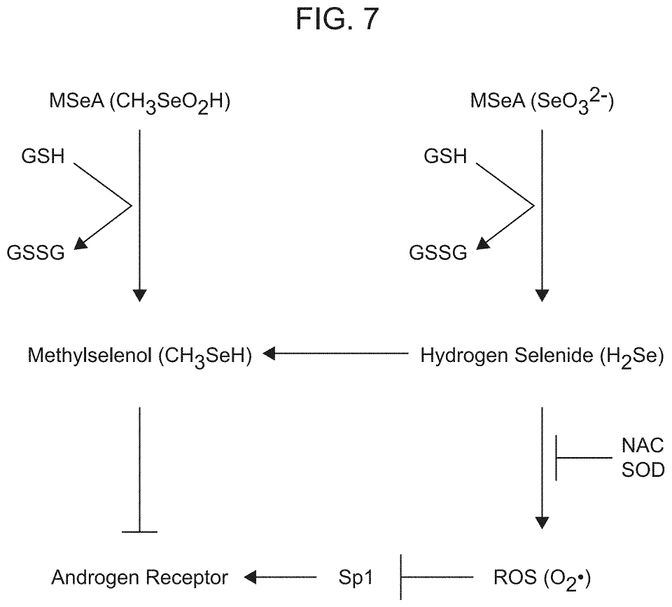

FIG. 8: Dosing simulations. (a) Single dose simulation and (b) proposed dose simulation using the final population PK model; desired systemic sodium selenite range demarcated by 395 and 790 mcg/L (lower and upper black dotted lines, respectively).

FIG. 9. Patient #2, a 76-year-old male with disseminated prostate carcinoma who received sodium selenite in combination with radiotherapy for metastases in the left shoulder and left hip. Tc-99m MDP bone scans pre (A) and 15 months post (B) treatment demonstrate near complete response in the left hip (open arrows) and partial response in the left shoulder (arrows).

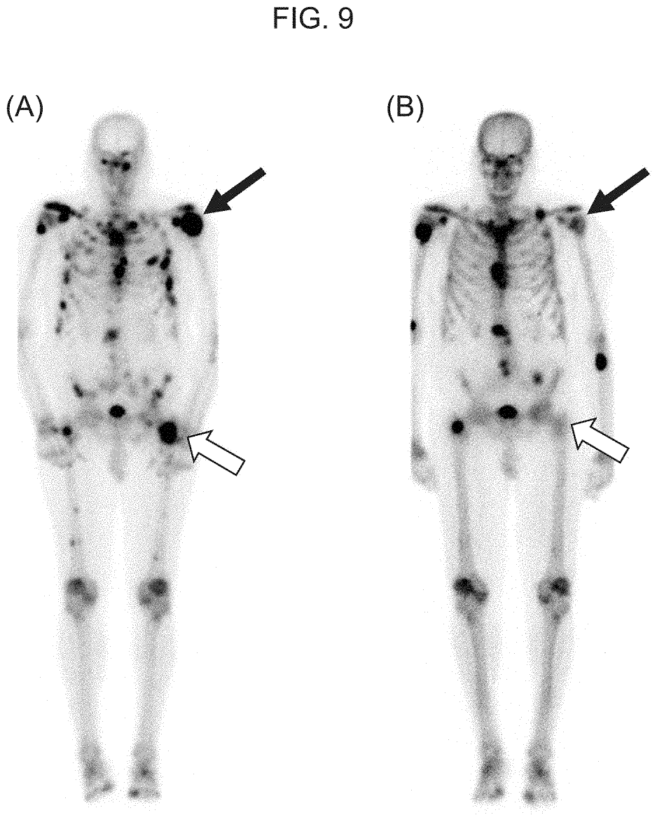

### DETAILED DESCRIPTION OF EMBODIMENTS

Methods for treating both androgen-responsive and unresponsive cancers using combination therapy with an inhibitor of the Sp1 transcription factor, radiation therapy, and/or androgen deprivation therapy are disclosed. Such combination treatment is especially useful for treating subjects who are resistant to androgen deprivation therapy, or who may become resistant to androgen deprivation therapy (e.g., those who have CRPC or other androgen-responsive cancer).

Before the present treatment methods are described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “an inhibitor” includes a plurality of such inhibitors and reference to “the inhibitor” includes reference to one or more inhibitors and equivalents thereof, e.g. drugs, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with cancer) as well as those in which prevention is desired (e.g., those with increased susceptibility to cancer, those suspected of having cancer, those with a risk of developing resistance to androgen deprivation therapy, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, Ill, or IV) or grade (G1, G2, G3, etc.) of neoplasia, tumor, or cancer, or a neoplasia, tumor, cancer or metastasis that is progressing, worsening, stabilized or in remission.

Cancers and cancer cells that can be treated include, but are not limited to, hematological cancers, including leukemia, lymphoma and myeloma, and solid cancers, including for example tumors of the brain (glioblastomas, medulloblastoma, astrocytoma, oligodendroglioma, ependymomas), carcinomas, e.g. carcinoma of the lung, liver, thyroid, bone, adrenal, spleen, kidney, small intestine, pancreas, colon, stomach, breast, endometrium, prostate, testicle, ovary, skin, head and neck, and esophagus.

In particular, the terms “tumor,” “cancer” and “neoplasia” include carcinomas, such as squamous cell carcinoma, adenocarcinoma, adenosquamous carcinoma, anaplastic carcinoma, large cell carcinoma, neuroendocrine carcinoma, and small cell carcinoma.

An “androgen-responsive cancer” includes any cancer that is responsive to androgenic hormones that bind to the androgen receptor, including testosterone and dihydrotestosterone, which promote cancer growth. Androgen-responsive cancers include, but are not limited to, androgen-responsive prostate cancer, breast cancer (e.g., triple-negative breast cancer), salivary gland cancer (e.g., salivary duct carcinoma), bladder cancer, and esophageal cancer (e.g., esophageal adenocarcinoma).

In certain embodiments, the androgen-responsive cancer is prostate cancer such as, but not limited to, prostate adenocarcinoma and includes other types of cancers (e.g., neuroendocrine, acinar, or ductal adenocarcinoma), transitional cell cancer, sarcoma, carcinoid, or small cell carcinoma. The prostate cancer may be of any type, stage or grade, and may have, for example, a Gleason score of 2 to 10. In certain embodiments, the prostate cancer is CRPC or castrate (or hormone)-refractory prostate cancer (i.e., prostate cancer resistant to androgen deprivation therapy).

By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models.

The term “survival” as used herein means the time from the start of treatment (i.e., combination therapy with an inhibitor of the Sp1 transcription factor and androgen deprivation therapy, as described herein) to the time of death.

By “therapeutically effective dose or amount” of an inhibitor of the Sp1 transcription factor (e.g., selenite), or a therapeutic agent used in androgen deprivation therapy (e.g., abiraterone) is intended an amount that, when the inhibitor of the Sp1 transcription factor and androgen deprivation therapy are administered in combination, as described herein, brings about a positive therapeutic response, such as anti-tumor activity and/or increased progression-free or overall survival.

The term “tumor response” as used herein means a reduction or elimination of all measurable lesions. The criteria for tumor response are based on the WHO Reporting Criteria [WHO Offset Publication, 48-World Health Organization, Geneva, Switzerland, (1979)]. Ideally, all uni- or bidimensionally measurable lesions should be measured at each assessment. When multiple lesions are present in any organ, such measurements may not be possible and, under such circumstances, up to 6 representative lesions should be selected, if available.

The term “complete response” (CR) as used herein means a complete disappearance of all clinically detectable malignant disease, determined by 2 assessments at least 4 weeks apart.

The term “partial response” (PR) as used herein means a 50% or greater reduction from baseline in the sum of the products of the longest perpendicular diameters of all measurable disease without progression of evaluable disease and without evidence of any new lesions as determined by at least two consecutive assessments at least four weeks apart.

“Substantially purified” generally refers to isolation of a substance (compound, drug, polynucleotide, protein, polypeptide) such that the substance comprises the majority percent of the sample in which it resides. Typically in a sample, a substantially purified component comprises 50%, preferably 80%-85%, more preferably 90-95% of the sample. Techniques for purifying substances of interest are well-known in the art and include, for example, ion-exchange chromatography, affinity chromatography and sedimentation according to density.

The terms “recipient”, “individual”, “subject”, “host”, and “patient” are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

The term “unit dosage form,” as used herein, refers to physically discrete units suitable as unitary dosages for human and animal subjects, each unit containing a predetermined quantity of the agents calculated in an amount sufficient to produce the desired effect in association with a pharmaceutically acceptable diluent, carrier or vehicle. The specifications for the unit dosage forms for use in the present invention depend on the particular compound employed and the effect to be achieved, the pharmacodynamics associated with each compound in the host, and the like.

The term “radiosensitizer,” as used herein, is defined as a compound administered to a human or other animal in a therapeutically effective amount to increase the sensitivity of cells to electromagnetic radiation and/or to promote the treatment of diseases that are treatable with electromagnetic radiation or where electromagnetic radiation has a palliative effect. Radiation therapy may include brachytherapy (use of radionuclides), electron beam therapy, alpha particle therapy, and targeted radionuclide therapy. The terms “electromagnetic radiation” and “radiation” as used herein include, but are not limited to, gamma and X-rays as used for therapeutic radiation. Preferred embodiments of the present invention employ the electromagnetic radiation of gamma-radiation (10⁻²⁰ to 10⁻¹³ m), X-ray radiation (10⁻¹² to 10⁻⁹ m), ultraviolet light (10 nm to 400 nm, visible light (400 nm to 700 nm), infrared radiation (700 nm to 1.0 mm), and microwave radiation (1 mm to 30 cm). Many cancer treatment protocols currently employ drugs (e.g. chemotherapeutic agents) that can be radiosensitizers for ionizing radiation.

Radiotherapy can be used with either curative or palliation intent to treat a wide range of malignancies. The distinction between curative and palliative goals may be blurred in some patients with cancer. Treatment recommendations are made be on the basis of many factors, including the patient's tumor type, stage, prior therapy, general state of health and preferences. Goals may include symptom relief at the site of primary tumor or from metastatic lesions. The WHO defines palliative care as “an approach that improves the quality of life of patients and their families facing the problems associated with life-threatening illness, through the prevention and relief of suffering by means of early identification and impeccable assessment and treatment of pain and other problems, physical, psychosocial, and spiritual.”

Historically, curative treatment schemes have been developed to deliver daily fraction sizes generally of 1.8 to 2.5 Gy to doses totaling between approximately 20 and 80 Gy (depending on tumor histology, stage, location, prior therapy, and sequential or concurrent use of chemotherapy or other systemic therapy). More recently, hypofractionated regimens are used in some clinical settings with fraction sizes of approximately 3-20 Gy in 1 or more fractions (usually in the range of 3-5). Palliative treatment courses of 8 to 30 Gy given in 1 to 10 fractions have been shown to be useful for a wide range of scenarios. For example, palliative treatment of bone metastases include acceptable fractionation schemes such as: 30 Gy in 10 fractions, 24 Gy in six fractions, 20 Gy in five fractions, 27 Gy in 3 fractions, 8 Gy in one fraction, etc.

Specifically for prostate cancer, systemic treatments, such as hormonal ablation/androgen deprivation therapy (ADT) play a major role in the treatment of patients with prostate cancer—both localized in conjunction with radiation therapy, and in the setting of metastatic/castrate resistant prostate cancer (CRPC). Palliative radiotherapy can also add greatly to the improvement of symptoms such as pain, obstruction, nerve root compression, etc. Patients with symptomatic sites of castration-resistant prostate cancer may achieve good symptom relief with radiotherapy courses using dose/fractionation regimens as above, but often the benefit of palliative radiation therapy is not complete or long lasting.

In this respect selenite can have multiple beneficial effects. Selenite can make radiation therapy more effective. It also downregulates the level of expression and function of the androgen receptor via inhibition of Sp1, which occurs via a redox mechanism.

The propensity of many primary tumor types to spread to the skeleton is high. Symptoms from bone metastases may commonly include pain, pathologic fracture, or spinal cord/nerve root compression. When combined with the appropriate use of other measures such as a pain medicine regimen, surgical stabilization, systemic treatments including bone-strengthening agents, and radiopharmaceuticals, external beam radiotherapy can be an efficacious and well-tolerated treatment for painful bone metastases. Fractionation schemes for bone metastases show pain relief equivalency for schedules including 30 Gy in 10 fractions, 24 Gy in six fractions, 20 Gy in five fractions, and a single 8-Gy fraction, although the duration of benefit may be shorter with a single fraction of radiation.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect.

“Concomittant administration” of a cancer therapeutic drug, antibody, tumor vaccine, hormone therapy, etc. means administration of the agents at such time that the agents each and sometimes simultaneously will have a therapeutic effect. Such concomitant administration may involve concurrent (i.e. at the same time), prior, or subsequent administration of the agents with respect to the administration of each other. A person of ordinary skill in the art would have no difficulty determining the appropriate timing, sequence and dosages of administration for particular drugs and compositions of the present invention.

As used herein, endpoints for treatment will be given a meaning as known in the art and as used by the Food and Drug Administration.

Endpoints that are based on tumor assessments include DFS, ORR, TTP, PFS, and time-to-treatment failure (TTF). The collection and analysis of data on these time-dependent endpoints are based on indirect assessments, calculations, and estimates (e.g., tumor measurements). Disease-Free Survival (DFS) is defined as the time from randomization until recurrence of tumor or death from any cause. The most frequent use of this endpoint is in the adjuvant setting after definitive surgery or definitive radiation therapy, e.g. with intention for curative therapy. DFS also can be an important endpoint when a large percentage of patients achieve complete responses with chemotherapy.

Overall survival is defined as the time from randomization until death from any cause, and is measured in the intent-to-treat population. Survival is considered the most reliable cancer endpoint, and when studies can be conducted to adequately assess survival, it is usually the preferred endpoint. This endpoint is precise and easy to measure, documented by the date of death. Bias is not a factor in endpoint measurement. Survival improvement should be analyzed as a risk-benefit analysis to assess clinical benefit. Overall survival can be evaluated in randomized controlled studies. Demonstration of a statistically significant improvement in overall survival can be considered to be clinically significant if the toxicity profile is acceptable, and has often supported new drug approval. A benefit of the methods of the invention can include increased overall survival of patients.

Objective Response Rate. ORR is defined as the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study.

Time to Progression and Progression-Free Survival. TTP and PFS have served as primary endpoints for drug approval. TTP is defined as the time from randomization until objective tumor progression; TTP does not include deaths. PFS is defined as the time from randomization until objective tumor progression or death. The precise definition of tumor progression is important and should be carefully detailed in the protocol.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

### Methods

Methods for treating androgen-responsive cancers using combination therapy with an inhibitor of the Sp1 transcription factor, radiotherapy, and/or androgen deprivation therapy are disclosed.

Without being bound by a particular theory, androgen resistance is thought to be mediated by increased expression and/or increased activity of the androgen receptor (i.e., hyperactive or constitutively active androgen receptor). AR splice variants 5, 6 and 7 are known to exhibit ligand-independent constitutive activity and sensitize the full-length androgen receptor to low ligand concentrations. The Sp1 transcription factor is needed to induce expression of the androgen receptor (both full-length and variant forms). Thus, any agent that inhibits the Sp1 transcription factor may overcome resistance to androgen deprivation therapy. Accordingly, combination therapy with an inhibitor of the Sp1 transcription factor and androgen deprivation therapy can be used to treat androgen-responsive cancers (e.g., prostate cancer) to delay or decrease the risk of developing androgen resistance, or to overcome resistance to androgen deprivation therapy once it has developed. While the methods of the invention are directed to treatment of an existing tumor, it is recognized that the methods may be useful in preventing the development of new lesions/tumors arising during therapy.

### Inhibitors of the Sp1 Transcription Factor

As explained above, the methods of the present invention include administering an inhibitor of the Sp1 transcription factor in combination with androgen deprivation therapy for treatment of an androgen-responsive cancer. Exemplary inhibitors of the Sp1 transcription factor for use in the methods described herein include selenite and other agents such as Withaferin A, mithramycin (aureolic acid, plicamycin) and analogues such as mithramycin SDK (SDK), mithramycin SK (SK), and premithrmycin B (PreB), 17-allylamino-17-demethoxygeldanamycin (17-AAG), EC-8042, bisanthracycline WP631, tolfenamic acid (clotam), tripartite motif 22 (TRIM22) peptide inhibitors, anti-sense, small interfering RNA (siRNA), or other nucleic acid inhibitors; and derivatives and analogues thereof. Such inhibitors inhibit binding of the Sp1 transcription factor to the AR gene promoter, which inhibits AR expression, and/or induce degradation of the Sp1 transcription factor.

For example, selenite including water soluble alkali metal salts thereof such as the sodium and potassium salts (i.e., sodium and potassium selenite) can be used. Selenite salts are commercially available, for example, from Millipore-Sigma (Burlington, Mass.), Spectrum Chemical (New Brunswick, N.J.), American Elements (Los Angeles, Calif.), and Santa Cruz Biotechnology, Inc. (Dallas, Tex.).

### Androgen Deprivation Therapy

Androgen deprivation therapy is administered in combination with the inhibitor of the Sp1 transcription factor (e.g. selenite). Androgen-responsive cancer cells depend on androgen hormones to grow. Androgens such as testosterone, dihydrotestosterone (DHT), and androstenedione are produced by the testicles and adrenal glands. Androgen deprivation therapy decreases the levels of these androgen hormones using drugs or surgery or interferes with the binding of the androgen hormones to the androgen receptor.

Examples of therapeutic agents that can be used in androgen deprivation therapy include, but are not limited to, luteinizing hormone-releasing hormone agonists and antagonists such as leuprolide, goserelin, triptorelin, histrelin, buserelin, and degarelix; CYP17 inhibitors such as abiraterone, ketoconazole, orteronel, galeterone, and seviteronel; anti-androgens such as cyproterone acetate, enzalutamide, apalutamide, flutamide, bicalutamide, and nilutamide; and other androgen-suppressing agents such as estrogen and derivatives and analogues thereof. Alternatively or additionally, androgen deprivation therapy may include surgical castration (i.e., orchiectomy) to remove the testicles where androgens are produced.

### Pharmaceutical Compositions

Pharmaceutical compositions comprising an inhibitor of a SP1 transcription factor and/or an androgen deprivation therapy therapeutic agent in accordance with the present invention may be provided in various physical forms, for a variety of methods and routes of administration. For example, compositions can be formulated for, for example, injectable (parenteral), oral, topical, mucosal, or suppository administration. Also of interest are compositions formulated for administration by intravenous, intramuscular, subcutaneous, intratumoral, tumor targeted, or peritumoral routes, as well as administration to the vasculature of a tumor bed. The inhibitor of the SP1 transcription factor and the androgen deprivation therapy therapeutic agent may be in the same or different compositions.

The compositions may comprise inert or active additives. For example, the compositions can further comprise a suitable pharmaceutically acceptable excipient, which may be a vehicle, carrier, diluent, and/or adjuvant. The compositions can further comprise pharmaceutically acceptable auxiliary substances, such as pH adjusting and buffering agents, tonicity adjusting agents, stabilizers, wetting agents and the like, are readily available to the public. The selection of such suitable additional components will depend upon, for example, the form desired, the route of administration, and the neoplastic disease to be treated. The additional components are generally selected so as to not detrimentally affect any of the active ingredients of the composition.

Exemplary inert carriers or vehicles include: sugars and milk sugars, such as lactose;

liquids, such as water, isotonic aqueous solutions, saline solutions and alcohol; and inert powders, creams, salves, ointments, cleansing and antiseptic agents and the like.

Exemplary pharmaceutically active additional components may include cytotoxic agents, e.g., chemotherapeutic drugs, biological response modifiers, or radiosensitizers. As used herein, the term “biological response modifier” (BRM) refers to compounds which are, in their naturally-occurring state, produced in small amounts as part of the body's natural response to cancer or other diseases. Exemplary BRMs include monoclonal antibodies that bind to an antigen of malignant cells, and which may have an attached cytotoxic molecule (e.g., toxin, radioactive component, etc.); and cytokines (e.g. interferons, interleukins, colony-stimulating factors (CSFs)), which can stimulate blood cell production and help restore blood cell counts more rapidly after treatment. BRMs can be isolated, naturally-occurring molecules or recombinantly or otherwise artificially produced. Examples of these drugs include, but not limited to Rituxan, anti-HER-2, anti-PMSA, CMA-676, IFN-α (e.g., IFN-α2a, IFN-α2b, consensus interferon), Interleukin-2, Interleukin-3, Erythropoetin, Epoetin, G-CSF, GM-CSF, Filgrastim, Sargramostim and Thrombopoietin, as well as modified forms (e.g., PEGylated and hyperglycosylated forms) of such molecules. See, e.g., U.S. Patent Application Publication No. 20020107225, incorporated by reference herein.

In the subject methods, the inhibitor of the SP1 transcription factor and the androgen deprivation therapy therapeutic agent may be administered to the host using any convenient means capable of resulting in the desired therapeutic effect. Thus, the agents can be incorporated into a variety of formulations for therapeutic administration. More particularly, the inhibitor of the SP1 transcription factor and the androgen deprivation therapy therapeutic agent, in combination with appropriate, pharmaceutically acceptable excipients (e.g., carriers or diluents), may be formulated into preparations in solid, semi-solid, liquid or gaseous forms, such as tablets, capsules, powders, granules, ointments, solutions, suppositories, injections, inhalants and aerosols. In general, the inhibitor of the SP1 transcription factor and the androgen deprivation therapy therapeutic agent for use in combination therapy as described herein are formulated for enteral administration (e.g., by oral, buccal, or rectal administration), or parenteral administration (e.g., by subcutaneous, intradermal, intraperitoneal, intravenous, or intramuscular administration, e.g., infusion or injection). Administration may also be accomplished by, for example, transdermal, intratracheal, or inhalation administration.

In pharmaceutical dosage forms, the inhibitor of the SP1 transcription factor and the androgen deprivation therapy therapeutic agent may be administered in the form of their pharmaceutically acceptable salts, or they may also be used alone or in appropriate association, as well as in combination, with other pharmaceutically active compounds. The following methods and excipients are merely exemplary and are in no way limiting.

The inhibitor of the SP1 transcription factor and the androgen deprivation therapy therapeutic agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

In the case of the injectable form of the inhibitor of the SP1 transcription factor and the androgen deprivation therapy therapeutic agent, the compounds may be dissolved in an aqueous buffer to form a parenteral preparation. Suitable aqueous buffers include, but are not limited to, acetate, succinate, citrate, phosphate buffers varying in strength typically from 5 mM to 100 mM, and distilled or sterilized water. In some embodiments, the aqueous buffer may include sodium chloride, and sugars e.g., mannitol, dextrose, sucrose, glucose and the like.

For oral preparations, the inhibitor of the SP1 transcription factor and the androgen deprivation therapy therapeutic agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Furthermore, the inhibitor of the SP1 transcription factor and the androgen deprivation therapy therapeutic agent can also be provided in sustained release or controlled release formulations, e.g., to provide for release of agent over time and in a desired amount (e.g., in an amount effective to provide for a desired therapeutic or otherwise beneficial effect).

Unit dosage forms for oral or rectal administration also include, for example, syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

It is to be understood that the particular carriers or vehicles set out above are illustrative only and other known pharmaceutically acceptable materials can be utilized in the compositions of this invention so long as they do not adversely react or interact with the inhibitor of the SP1 transcription factor and the androgen deprivation therapy therapeutic agent and other active ingredients to destroy the identity or activity thereof. Moreover, the particular carrier or vehicle chosen for use will depend upon the form of the composition needed for the particular method of administration and the host to receive the composition.

In those cases, where the composition contains a larger amount of a compound (e.g., in some embodiments more than about 1.0 mg by weight), the composition may be employed in the form of divided dosages when being administered whether it be in the form of a tablet, a capsule or a liquid solution. Moreover, a particular dosage in this respect can be administered several times a day so long as the total amount of the compound does not exceed a generally accepted maximum dosage.

In some instances, the composition can be made by simply mixing the compound(s) (i.e., the inhibitor of the SP1 transcription factor and/or the androgen deprivation therapy therapeutic agent) in proper proportion with an appropriate carrier. For example, in preparing tablets, an alkali metal selenite or selenate salt in its dry form may be mechanically mixed with a powdered carrier or vehicle and shaped or pressed into tablets or encapsulated with or without the androgen deprivation therapy therapeutic agent by known art recognized techniques. On the other hand, if desirable, such salts can be dissolved in water and then mixed with a powdered carrier and shaped or pressed into tablets.

As an alternative, liquid compositions can be prepared simply by dissolving the inhibitor of the SP1 transcription factor and/or the androgen deprivation therapy therapeutic agent in water and using the composition in that form with recognized additives for either external or oral application. The materials as mixed should contain a desired amount of the inhibitor of the SP1 transcription factor and/or the androgen deprivation therapy therapeutic agent, which in a single or divided dose achieve a desired therapeutic effect.

### Administration

Combination therapy with an inhibitor of the SP1 transcription factor (e.g., selenite) and androgen deprivation therapy (e.g., abiraterone or enzalutamide) will be administered to a subject having an androgen-responsive cancer. Combination therapy of selenite and radiotherapy, which may be palliative radiotherapy, can be performed on an androgen-responsive cancer such as prostate cancer, but need not be limited to such. For example, cancers resulting in bone metastases may be treated with palliative radiation therapy, and include solid tumors such as carcinomas, sarcomas, glioblastomas, melanomas, lymphomas, myelomas, etc., and circulating cancers such as leukemias. Examples of cancer include but are not limited to, ovarian cancer, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer.

Androgen-responsive cancers include, but are not limited to, androgen-responsive prostate cancer, breast cancer (e.g., triple-negative breast cancer), salivary gland cancer (e.g., salivary duct carcinoma), bladder cancer, and esophageal cancer (e.g., esophageal adenocarcinoma). In some embodiments, the androgen-responsive cancer is prostate cancer, such as prostate adenocarcinoma (e.g., neuroendocrine, acinar, or ductal adenocarcinoma), neuroendocrine prostate cancer, transitional cell cancer, sarcoma, carcinoid, or small cell carcinoma. In certain embodiments, the prostate cancer is CRPC or hormone-refractory prostate cancer (i.e., prostate cancer resistant to androgen deprivation therapy). Combination therapy with an inhibitor of the SP1 transcription factor and androgen deprivation therapy, as described herein, decreases androgen receptor and/or androgen hormone expression or activity levels and keeps them low enough to prevent tumor growth, and delays or decreases the risk of developing resistance to treatment. This combination therapy may also overcome/reverse resistance to androgen deprivation therapy that has developed before the addition of selenite to the regimen.

At least one therapeutically effective dose of an inhibitor of the SP1 transcription factor, e.g. selenite is administered in combination with one or both of radiotherapy and an androgen deprivation therapy. By “therapeutically effective dose or amount” of each of these agents is intended an amount that when administered in combination, brings about a positive therapeutic response with respect to treatment of an individual for cancer, for example, including, without limitation prostate cancer, and including CRPC, breast cancer, salivary gland cancer, bladder cancer, and esophageal cancer.

Of particular interest is an amount of these agents that provides an anti-tumor effect, as defined herein and/or increases freedom from progression or overall survival, and/or reduces pain and stabilizes metastases. By “positive therapeutic response” is intended the individual undergoing the combination treatment according to the invention exhibits a stabilization of disease or an improvement in one or more symptoms/signs of the androgen-responsive cancer for which the individual is undergoing therapy, such as one or more of the following improvements in the disease: (1) reduction in tumor size; (2) reduction in the number of cancer cells; (3) inhibition (i.e., slowing to some extent, preferably halting) of tumor growth; (4) inhibition (i.e., slowing to some extent, preferably halting) of cancer cell infiltration into peripheral organs; (5) inhibition (i.e., slowing to some extent, preferably halting) of tumor metastasis; (6) decrease in serum levels of PSA, and/or (7) some extent of relief from one or more symptoms associated with the cancer, such as pain from bone metastases.

Such therapeutic responses may be further characterized as to degree of improvement. Thus, for example, an improvement may be characterized as a complete response. By “complete response” is documentation of the disappearance of all symptoms and signs of all measurable or evaluable disease confirmed by physical examination, laboratory, nuclear and radiographic studies (i.e., CT (computer tomography) and/or MRI (magnetic resonance imaging)), and other non-invasive procedures repeated for all initial abnormalities or sites positive at the time of entry into the study. Alternatively, an improvement in the disease may be categorized as being a partial response. By “partial response” is intended a reduction of greater than 50% in the sum of the products of the perpendicular diameters of all measurable lesions when compared with pretreatment measurements (for patients with evaluable response only, partial response does not apply).

In certain embodiments, multiple therapeutically effective doses of an agent or agents will be administered according to a daily dosing regimen or intermittently. For example, a therapeutically effective dose can be administered once daily, twice daily, three times daily, or one day a week, two days a week, three days a week, four days a week, five days a week, six days a week, or seven days a week, and so forth. By “intermittent” administration is intended the therapeutically effective dose can be administered, for example, every other day, every two days, every three days, and so forth. For example, in some embodiments, the inhibitor of the SP1 transcription factor will be administered once a day, twice a day, three times a day, and so forth. The androgen deprivation therapy therapeutic agent may be administered according to the standard of care, such as monthly, weekly, twice-weekly, thrice-weekly, or daily for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of the agent in question are administered to the subject within a 7-day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7-day period, allowing for a minimum of 48 hours between doses and a maximum of 72 hours between doses. For purposes of the present disclosure, this type of dosing is referred to as “intermittent” therapy. In accordance with the methods disclosed herein, a subject can receive intermittent therapy (i.e., twice-weekly or thrice-weekly administration of a therapeutically effective dose) for one or more weekly cycles until the desired therapeutic response is achieved, and may continue to be administered as maintenance therapy thereafter. The agents can be administered by any acceptable route of administration as noted herein below.

The inhibitor of the SP1 transcription factor can be administered prior to, concurrent with, or subsequent to the androgen deprivation therapy and/or radiotherapy. If provided at the same time as the androgen deprivation therapy, the inhibitor of the SP1 transcription factor can be provided in the same or in a different composition than an androgen deprivation therapy therapeutic agent. Thus, the agents can be presented to the individual by way of concurrent therapy. By “concurrent therapy” is intended administration to a human subject such that the therapeutic effect of the combination of the substances is caused in the subject undergoing therapy. For example, concurrent therapy may be achieved by administering at least one therapeutically effective dose of a pharmaceutical composition comprising an inhibitor of the SP1 transcription factor and at least one therapeutically effective dose of a pharmaceutical composition comprising at least one androgen deprivation therapy therapeutic agent according to a particular dosing regimen. Administration of the separate pharmaceutical compositions can be at the same time (i.e., simultaneously) or at different times (i.e., sequentially, in either order, on the same day, or on different days), as long as the therapeutic effect of the combination of these substances is caused in the subject undergoing therapy.

In certain embodiments, treatment with the inhibitor of the SP1 transcription factor is continued for a period after androgen deprivation therapy is discontinued in order to ensure that androgen receptor and hormone levels remain low enough to prevent tumor growth and delay or decrease the risk of developing resistance to treatment. For example, treatment with the inhibitor of the SP1 transcription factor can be continued after surgical castration of a patient or treatment with one or more androgen deprivation therapy therapeutic agents.

In other embodiments of the invention, the pharmaceutical compositions comprising the agents such as the inhibitor of a SP1 transcription factor and an androgen deprivation therapy therapeutic agent are a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The pharmaceutical compositions comprising the inhibitor of the SP1 transcription factor and the androgen deprivation therapy therapeutic agent may be administered using the same or different routes of administration in accordance with any medically acceptable method known in the art. Suitable routes of administration include parenteral administration, such as subcutaneous (SC), intraperitoneal (IP), intramuscular (IM), and intravenous (IV), or infusion, oral, pulmonary, nasal, topical, transdermal, and suppositories. Also of interest is administration by intratumoral, tumor targeted, or peritumoral routes, as well as administration to the vasculature of a tumor bed. Where the composition is administered via pulmonary delivery, the therapeutically effective dose is adjusted such that the soluble level of the agent, such as the inhibitor of the SP1 transcription factor or androgen deprivation therapy therapeutic agent in the bloodstream, is equivalent to that obtained with a therapeutically effective dose that is administered orally or parenterally, for example SC, IP, IM, or IV. In some embodiments, the therapeutic agents are administered locally at the site of a tumor. For example, the inhibitor of the SP1 transcription factor and/or the androgen deprivation therapy therapeutic agent may be injected into a tumor or administered on a patch or in a gel.

Factors influencing the respective amount of the various compositions to be administered include, but are not limited to, the mode of administration, the frequency of administration (i.e., daily, or intermittent administration, such as twice- or thrice-weekly), the particular androgen-responsive cancer undergoing therapy, the severity of the disease, the history of the disease, the extent of resistance to androgen deprivation therapy, history of prior therapy, the particular therapeutic agents administered, whether the individual is undergoing concurrent therapy with an additional therapeutic agent, and the age, height, weight, health, and physical condition of the individual undergoing therapy. A higher dosage of an agent may be preferred with increasing weight of the subject undergoing therapy.

In order to achieve efficacy, the blood levels of the therapeutic agents are maintained above a specific level for a specific time. Efficacy is dose dependent and higher blood levels of the therapeutic agents may further decrease androgen receptor and/or androgen hormone expression or activity levels and contribute to greater anti-tumor effects. In order to minimize toxicity, the levels of the therapeutic agents in the blood may be kept within a certain range, and/or below a certain level within a specific time and for a specific time (i.e., a “rest period” that allows clearance of the therapeutic agents). That is, the therapeutic agents are kept within a certain range or below a certain level for a certain time before the next dose is given.

In certain embodiments, the method of treatment of a patient having an androgen-responsive cancer comprises a treatment cycle with an inhibitor of a Sp1 transcription factor in combination with androgen deprivation therapy followed by a rest period in which no inhibitor of the Sp1 transcription factor and/or the androgen deprivation therapy therapeutic agent is administered to allow the patient to “recover” from the undesirable effects of these agents. Multiple doses of the inhibitor of the Sp1 transcription factor and/or the androgen deprivation therapy therapeutic agent can be administered according to a daily dosing regimen or intermittently, followed by a rest period. Thereafter, a new schedule of dosing may be administered to keep androgen receptor and/or androgen hormone levels low enough to prevent cancer growth. Treatment with the inhibitor of the Sp1 transcription factor may be continued following courses or cycles of androgen deprivation therapy to ensure the desired combination effect.

Where a subject undergoing therapy in accordance with the previously mentioned dosing regimens exhibits a partial response, or a relapse following a prolonged period of remission, subsequent courses of concurrent therapy may be needed to achieve stabilization of disease or complete remission of the disease. Thus, subsequent to a period of time off from a first treatment period, a subject may receive one or more additional treatment periods comprising an inhibitor of a Sp1 transcription factor in combination with androgen deprivation therapy. Such a period of time off between treatment periods is referred to herein as a time period of discontinuance. It is recognized that the length of the time period of discontinuance is dependent upon the degree of tumor response (i.e., complete versus partial) achieved with any prior treatment periods of concurrent therapy with these therapeutic agents.

In an exemplary embodiment, the inhibitor of the Sp1 transcription factor is selenite, which can be administered in an amount of, for example, about 16.5 mg daily, 33 mg daily, 49.5 mg daily, 66 mg daily, 99 mg daily, or 121 mg daily, or more frequently (e.g. two to three times/day) with the proviso that a desirable therapeutic index is achieved. In some embodiments, selenite is administered in amounts ranging from approximately 5 mg, 10 mg, 15 mg, 20 mg, 25 mg, to 100 mg daily, as single or divided doses, e.g. twice daily. Doses of selenite may be greater than that normally associated with use of selenite as a chemopreventive agent, and can be greater than that used in supportive cancer therapy (in which selenite is administered as a protective agent for normal cells in cancer therapy, but not as an anti-cancer agent itself). For example, selenite compounds can be administered at doses greater than 200 μg per day (e.g., for a 75 kg individual, usually by oral administration).

Doses of selenite, in accordance with the treatment methods described herein, can be administered in whole or divided doses, and can may be administered daily (e.g., once a day or in divided doses—e.g. two times a day), thrice weekly, twice weekly, weekly, and the like, with daily or weekly dosing being of particular interest. It will be appreciated that the amounts of selenite administered will vary with a variety of factors including, but not limited to, the type of radiotherapy, the type of androgen deprivation therapy (e.g., luteinizing hormone-releasing hormone (LHRH) agonist, a CYP17 inhibitor, an anti-androgen, or other androgen-suppressing drug), form of selenite administered (e.g., selenite or type of salt of selenite, and the like), route of administration, formulation, dosage form, severity or extent of disease, tumor type (e.g., localized, metastatic, tissue or origin, and the like), extent of resistance to androgen deprivation therapy, the age, height, weight, health, and physical condition of the subject, and other factors that will be readily appreciated by a clinician or other health care practitioner.

In another exemplary embodiment, androgen deprivation therapy comprises administering the therapeutic agent, abiraterone (Zytiga). Typically, abiraterone is administered in an amount of 125 mg to about 1000 mg daily, including any dosage in this range such as 125 mg, 250 mg, 500 mg, or 1000 mg, though usually about 500 mg to about 1000 mg is self-administered orally once a day (other modes of administration or frequencies of dosing can be used). In some embodiments, the amount of abiraterone administered is greater than 1000 mg per day. Dosages may need to be reduced in patients with moderate liver dysfunction. It will be appreciated that the amounts of abiraterone administered will vary with a variety of factors including, but not limited to, the route of administration, formulation, dosage form, severity or extent of disease, tumor type (e.g., localized, metastatic, and the like), extent of resistance to androgen deprivation therapy, the age, height, weight, health, and physical condition of the subject, and other factors that will be readily appreciated by a clinician or other health care practitioner. Abiraterone will generally be administered according to the prescribed FDA-approved standard of care.

The combination therapy disclosed herein may be combined with other anti-cancer treatment regimens, including one or more other anti-cancer therapies such as surgery, chemotherapy, radiation therapy, immunotherapy/biological therapy, hormonal therapy, cryotherapy, high-intensity focused ultrasound (HIFU), and photodynamic therapy. For example, patients having prostate cancer with systemic disease after prostatectomy or radiation therapy may be further treated with palliative radiation therapy, chemotherapy (e.g., docetaxel, mitoxantrone and prednisone), systemic radiation therapy (e.g., samarium, strontium, Radium-223, or radiolabeled anti-PSMA or radiolabeled PSMA ligand) and/or anti-androgen therapy (e.g., surgical castration, finasteride, dutasteride). Patients with elevated PSA following prostatectomy who do not have systemic disease may be further treated with localized adjuvant therapy (e.g., radiation therapy of the prostate bed +/−pelvis lymph nodes) and/or a course of anti-androgen therapy. Alternatively or additionally, HIFU, which uses ultrasound rather than radiation to destroy cancerous cells, may be used. HIFU, which is minimally invasive, utilizes targeted sound waves focused on cancerous tissue. For treatment of prostate cancer, the ultrasound sound waves are typically transmitted through the rectal wall and focused at locations within the prostate that have been identified as being cancerous previously by imaging (e.g., MRI and/or ultrasound). Another possible anti-cancer treatment is cryotherapy (i.e., cryosurgery or cryoablation), which uses extreme cold to destroy cancer cells. For example, liquid nitrogen or argon can be applied locally directly on abnormal tissue with a cotton swab or spraying device to kill cancerous cells.

### Kits

Also provided are kits for treating a patient for an androgen-responsive cancer using combination therapy with an inhibitor of the SP1 transcription factor and an androgen deprivation therapy therapeutic agent as described herein. The inhibitor of the SP1 transcription factor and the androgen deprivation therapy therapeutic agent may be contained in separate compositions or in the same composition. Kits with unit doses of the formulations comprising the inhibitor of the SP1 transcription factor and/or the androgen deprivation therapy therapeutic agent suitable for use in the treatment methods described herein, e.g., in tablets or injectable dose(s), are provided. In such kits, in addition to the containers containing the unit doses will be an informational package insert describing the use and attendant benefits of the combination therapy for treating an androgen-responsive cancer. The kit can include, for example, a dosing regimen for the combination therapy with the inhibitor of the SP1 transcription factor and the androgen deprivation therapy therapeutic agent.

Formulations suitable for intravenous administration are of particular interest, and in such embodiments the kit may further include a syringe or other device to accomplish such administration, which syringe or device may be pre-filled with the inhibitor of the SP1 transcription factor and/or the androgen deprivation therapy therapeutic agent. The instructions can be printed on a label affixed to the container or can be a package insert that accompanies the container.

In certain embodiments, the inhibitor of the SP1 transcription factor included in the kit is selenite. A subject kit may include a container comprising a solution comprising a unit dose of selenite (e.g., sodium selenite), and a pharmaceutically acceptable excipient; and instructions to administer a unit dose according to a desired regimen or exemplary regimen dependent upon tumor type, age, weight, type of androgen deprivation therapy used in combination, and the like.

In certain embodiments, the androgen deprivation therapy therapeutic agent included in the kit is a CYP17 inhibitor (e.g., abiraterone, ketoconazole, orteronel, galeterone, or seviteronel). A subject kit may include a container comprising tablets or a solution comprising a unit dose of the CYP17 inhibitor, and a pharmaceutically acceptable excipient; and instructions to administer a unit dose according to a desired regimen or exemplary regimen dependent upon tumor type, age, weight, type of androgen deprivation therapy used in combination, and the like.

It will be apparent to one of ordinary skill in the art that various changes and modifications can be made without departing from the spirit or scope of the invention.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention.

### EXAMPLE 1

### Sp1 Inhibition by Selenite: Novel Treatment for Abiraterone-Resistant Castration-Resistant Prostate Cancer

Specificity protein 1 (Sp1) is a transcription factor for many genes essential for the regulation of multiple aspects of tumor cell survival, growth and angiogenesis. In prostate cancer cells, Sp1 is an essential transcriptional factor for AR expression. In addition, Sp1 interacts with AR, and acts as a co-activator for ligand-activated AR. Inhibition of Sp1 leads to decreased AR^{FL} and AR^{V567} expression and AR transcriptional activity, suppression of CRPC growth, and sensitization of CRPC cells to radiation and chemotherapy.

Abiraterone resistance (AbR) is thought to be mediated by increased expression of androgen receptor (AR) splice variants 5, 6 and 7, which have ligand-independent constitutive activity, and sensitize full-length AR to low ligand concentrations. Given that AR splice variants are important drivers of the development of AbR, we hypothesize that inhibitors of the transcription factor, Sp1, which is essential for expression of both full-length AR and variant forms of AR, will overcome AbR in CRPC and may be able to delay or decrease the risk of developing AbR CRPC.

Here we describe methods of using selenite for the treatment of prostate cancer. The metabolism of selenite depletes cells of an important antioxidant, glutathione (GSH), and results in the formation of superoxide, a highly reactive and toxic radical that results in the generation of reactive oxygen species. Our preclinical studies have demonstrated that 1) prostate cancer cells are more sensitive to selenite-induced apoptosis than normal prostate epithelial cells; 2) selenite induces significant growth inhibition of well-established prostate cancer tumors in mice at doses that have no detectable toxicity when administered both i.p. and p.o.; and 3) selenite is a potent radiosensitizer of both hormone sensitive and CRPC cells in vitro and in vivo.

Furthermore, in mechanistic studies, we found that selenite can inhibit AR expression and activity in LAPC-4 and LNCaP prostate cancer cells (Husbeck et al. (2006) Mol. Cancer Ther. 5(8):2078-2085). The Sp1 binding site in the AR promoter is a key regulatory component for its expression. In our experiments, selenite decreased Sp1 DNA binding and its expression in the nucleus. Furthermore, treatment of mice with selenite resulted in downregulation of AR expression in vivo, and decreased levels of PSA (downstream of Sp1) that correlated with tumor growth inhibition in both well-established hormone sensitive and CRPC tumors.

Patients with CRPC have a very poor prognosis and treatment options for these patients are limited. AbR is becoming an important and growing clinical problem. Targeting aberrant AR signaling with a Sp1 inhibitor is an entirely novel approach for the treatment of AbR. CRCP. Furthermore, Sp1 inhibitors should also be effective for treating resistance to enzalutamide, an inhibitor of AR signaling, that is upstream of Sp1. As a practical matter, the current formulation of selenite we used in our phase 1 trial is a simple formulation with a half-life of approximately 19 hours. A sustained release formulation can be used to enable convenient dosing and to maintain therapeutic blood levels for 12-24 hours for use in combination with abiraterone.

### EXAMPLE 2

### A Phase I Study Evaluating the Efficacy and Safety of Sodium Selenite in Combination with Abiraterone in Patients with Castrate-Resistant Prostate Cancer Progressing on Abiraterone

A phase 1 study is initiated to evaluate the efficacy and safety of sodium selenite in combination with Abiraterone in patients who have castrate-resistant prostate cancer (CRPC), undergoing treatment with Abiraterone, whose cancer is progressing on Abiraterone.

The data provided herein demonstrates that selenium, in the form of inorganic sodium selenite, is useful for treating both hormone sensitive and CRPC prostate cancer. This is based on data showing that 1) prostate cancer cells are more sensitive to selenium (sodium selenite)-induced apoptosis than normal prostate epithelial cells; 2) selenite induces significant growth inhibition of well-established prostate cancer tumors in mice at doses that have no detectable toxicity; and 3) sodium selenite radiosensitizes prostate tumors in vivo in xenograft models. Importantly, and relevant to this example, selenite disrupts AR signaling, and the inhibition of AR expression and activity by selenite occurs via a redox mechanism involving GSH, superoxide, and Sp1. Altogether, these findings suggest that selenium is useful in a variety of potential indications for prostate cancer, including both hormone sensitive and castrate-resistant prostate cancer, as a single agent, or in combination with radiation, chemotherapy or conventional hormone therapy. Selenite, as a novel inhibitor of AR expression and function in prostate cancer, can be used to prevent or overcome resistance to androgen deprivation therapy (e.g. Abiraterone).

Treatment Plan. In this study, sodium selenite is given orally (p.o.) three times a day. Planned dose levels include 5.5, 11.0, 16.5, 22.0, 27.5 and 33 mg, with and without food. Endpoints will include PK, toxicity with determination of MTD, and efficacy. An adaptive trial design is utilized.

Patients will take Abiraterone 1000 mg p.o. daily with prednisone 5 mg p.o. twice daily. Patients are maintained on Abiraterone, and sodium selenite is given concurrently with the Abiraterone.

### Example 3

Sodium Selenite Preparation. Sodium selenite pentahydrate (Active Pharmaceutical Ingredient (API)) is manufactured by Biosyn Arzneimittel GmbH in Fellbach, Germany, and the tablets are manufactured by Rottendorf Pharma GmbH in Ennigerloh, Germany. The drug is available in 5.5 mg tablets.

### Example 4

### Results of Stanford Phase 1 Trial of Sodium Selenite in Patients Receiving Palliative Radiation Therapy and Rationale for Proposed Dosing of Selenite

A Phase 1 trial of sodium selenite was performed in patients with advanced/metastatic cancer receiving palliative radiation therapy, the majority of which had CRPC. The primary objective of the study was to assess the safety and tolerability of this combination therapy. Secondary objectives included measurement of pharmacokinetics and evaluation of efficacy. The underlying hypothesis of this study was that the combination of selenite and radiation therapy would be safe and tolerable, and has the potential to improve PSA responses in the subset of patients with CRPC and local response to radiation therapy in patients with advanced/metastatic cancer.

The starting dose was 5.5 mg po/day with subsequent planned dose levels of 11, 16.5, 33, 49.5, 66, 99 and 121 mg po daily. Fifteen patients were treated with doses of sodium selenite ranging from 5.5 to 49.5 mg po/day 2 hours prior to scheduled radiation therapy treatments. Selenite was taken on an empty stomach (no food for 2 hours before or after). There was no toxicity attributable to selenite at the 5.5, 11 or 16.5 mg dose levels. Patients at all other dose levels experienced grade 1 and 2 GI toxicities. The use of ondansetron and loperamide-Hcl controlled the symptoms well for most patients. Most GI toxicities were grade 1, with the one patient treated at the 49.5 mg dose level having both grade 2 nausea/vomiting and diarrhea. Non-GI toxicities consisted of grade 1 fatigue and grade 1 QT prolongation in 2 patients, interpreted as within the range of expected intra-subject variability.

Using parameter estimates from a population PK model, the half-life was calculated to be approximately 18.5 hours. Cmax occurred at approximately 4 hours, but varied with dose level as follows. For the 5.5 mg dose level, Cmax (0.214 mg/L) occurred at 3.65 hours, with Area Under the Curve (AUC) 3.699 mg·hour/L. At the 11 mg dose level, Cmax (0.301 mg/L) occurred at 4.05 hours, with AUC 6.249 mg.hour/L. At 16.5 mg selenite, Cmax (0.366 mg/L) was at 4.2 hours, with AUC 8.365 mg.hour/L. At the 33 mg dose level, Cmax (0.497 mg/L) was at 4.35 hours, with AUC 13.285 mg.hour/L. Lastly, at 49.5 mg, Cmax (0.595 mg/L) was at 4.4 hours, with AUC of 16.904 mg.hour/L. Maximal blood levels of selenium were approximately 6.5-7.5 μM at the 33-49.5 mg dose level. This is in the range at which there was good activity in in vitro experiments.

Enrollment in this Phase 1 study was stopped because it was felt that the toxicity at the 33 mg dose level was well tolerated by patients and potentially clinically meaningful blood levels were obtained at that dose. The maximum tolerated dose (MTD) was not reached because it did not seem reasonable to subject this patient population to additional potential toxicity. In terms of efficacy, while most patients had improvement in their pain inventory scores, many with CRPC had a decrease in PSA, and some patients had responses on imaging although patient numbers were small, and the patient population heterogeneous. PK and toxicity data from this study was used to inform the planned study of selenite in combination with Abiraterone.

### Example 5

### Results from a Phase 1 Study of Sodium Selenite in Combination with Palliative Radiation Therapy in Patients with Metastatic Cancer

In preclinical studies, selenite had single agent activity and radiosensitized tumors in vivo. Here we report results from a Phase 1 trial in 15 patients with metastatic cancer treated with selenite (5.5 to 49.5 mg) orally as a single dose 2 hours before each radiation therapy (RT) treatment. Patients received RT regimens that were standard of care. The primary objective of the study was to assess the safety of this combination therapy. Secondary objectives included measurement of pharmacokinetics (PK) and evaluation of efficacy. Endpoints included assessment of PK, toxicity, tumor response, and pain before and after treatment. The half-life of selenite was 18.5 hours. There were no adverse events attributable to selenite until the 33 mg dose level, at which the primary toxicities were grade 1 GI side effects. One patient treated with 49.5 mg had grade 2 GI toxicity. Although this was not a DLT, it was felt that the highest acceptable dose in this patient population was 33 mg. Most patients had stabilization of disease within the RT fields, with some demonstrating objective evidence of tumor regression. Most patients had a marked improvement in pain and seven out of nine patients with prostate cancer had a decrease in PSA ranging from 11-78%. Doses up to 33 mg selenite were well tolerated in combination with RT.

Patients with metastatic cancer frequently receive palliative radiation therapy to treat painful and symptomatic sites of disease. Despite recent advances in both systemic and local treatment of metastases, many patients have persistent pain or symptoms following treatment. New and improved therapies are needed to increase the efficacy and duration of response to palliative radiation therapy.

Although a large body of data exists from studies of the potential utility of selenium supplementation (using an organic form of selenium) as a chemopreventive strategy, little is known regarding the use of selenium, as inorganic sodium selenite, as a cancer therapy. Our results demonstrate that selenium in the form of selenite can be used to treat prostate as well as other types of cancer. Importantly, selenite is metabolized differently than organic forms of selenium, with the key difference being that the metabolism of selenite depletes cells of an important antioxidant, glutathione (GSH), and results in the generation of superoxide, a highly reactive and toxic radical that results in the generation of reactive oxygen species (ROS).

Our work initially focused on prostate cancer. The rationale for using selenite to treat prostate cancer came from our preclinical studies showing that 1) prostate cancer cells are more sensitive to sodium selenite-induced apoptosis than normal prostate epithelial cells, 2) Selenite induces significant growth inhibition of well-established prostate cancer tumors in mice at doses that have no detectable toxicity when administered both ip and po, and 3) Selenite disrupts androgen receptor (AR) signaling, with inhibition of AR expression and activity by selenite occurring via a redox mechanism involving GSH, superoxide, and transcription factor Sp1. Altogether, these findings suggest that selenite may be useful in a variety of indications in the natural history of prostate cancer, including both hormone sensitive and hormone refractory prostate cancer, as a single agent, or in combination with radiation, chemotherapy or conventional androgen deprivation therapy (ADT).

Given that depletion of GSH is known to have radiosensitizing effects, and generation of superoxide should enhance the efficacy of radiation-induced ROS, selenite has the potential to sensitize a wide range of tumor types. Our data suggests that selenite-mediated tumor-selective radiosensitization in prostate cancer is due, in part, to differences between MnSOD and Bcl-2 family member expression in tumor vs. normal tissue. Similar differences in other tumor types, as well as overexpression of Nrf2 and its downstream target genes in cancer, may also contribute to the differential sensitizing effects of selenite.

In the Phase 1 trial described here, sodium selenite (given orally at daily doses of 5.5, 11, 16.5, 33 and 49.5 mg) was given concurrently with palliative radiation therapy in patients with metastatic cancer. The primary objective of the study was to assess the safety and tolerability of this combination therapy. Secondary objectives included measurement of pharmacokinetics and evaluation of efficacy. The underlying hypothesis of this study was that the combination of selenite and radiation therapy would be safe and tolerable, and have the potential to improve PSA responses in the subset of patients with castration-resistant prostate cancer (CRPC) and local response to radiation therapy in patients with metastatic cancer.

### Methods:

Patients: This study was approved by the Food and Drug Administration (IND 122151), the Stanford University Internal Review Board, and the Scientific Review Committee for the Stanford Cancer Institute. Fifteen patients with a variety of malignancies were treated on this study. The study was initially open only to prostate cancer patients, but then expanded to include a variety of tumor types. Patient characteristics are summarized in Table 1, including tumor histology, race, sex, age, BSA and history of prior therapy. Patients ranged in age from 37 to 92 years of age, with 13 men and two women. Before study entry, patients had to meet a number of eligibility criteria. Inclusion criteria included a) histologically-confirmed solid tumor malignancy with confirmation of metastasis, multiple myeloma, or plasmacytoma, b) need for palliative radiation therapy, c) for prostate cancer patients, PSA at least 2 ng/mL, except for patients who had recently started androgen deprivation therapy with PSA less than 2 ng/mL, d) age 8 years, e) life expectancy greater than 3 months, f) Eastern Cooperative Oncology Group (ECOG) performance status of zero or one or Karnofsky performance status 80%, and g) QT interval corrected using Fridericia's method (QTcF) <460 msec.

Exclusion criteria included a) Absolute neutrophil count <1500/μL, platelet count 100×10⁹/L, serum creatinine >2.0 mg/dL, total bilirubin >1.5× upper limit of normal (ULN), AST, and/or ALT >2× ULN, hemoglobin <9 g/dL, b) history of other malignancies within 5 years prior to Day 1 except for tumors that in the opinion of the investigators have a negligible risk for metastasis or death, such as adequately controlled basal cell carcinoma, squamous-cell carcinoma of the skin, or early-stage bladder cancer, c) current, or recent (within 4 weeks of the first treatment of this study) cytotoxic chemotherapy (eg, cisplatin, Taxol) or experimental drug therapy, d) uncontrolled inter-current illness, or psychiatric illness/social situations that would limit compliance with study requirements, e) history of myocardial infarction or unstable angina within 6 months prior to study enrollment, f) history of stroke or transient ischemic attack within 6 months prior to study enrollment, g) known human immunodeficiency virus (HIV) positivity while receiving antiretroviral therapies, and i) pregnant or breastfeeding women.

TABLE 1
Patient Characteristics

| | Dose | | | | | | |
|---|---|---|---|---|---|---|---|
| Patient | Cohort | Tumor | | | Age | | Prior |
| No. | (mg) | Histology | Race | Sex | (y) | BSA(m²) | Therapy |
| 1 | 5.5 | Prostate | White | M | 92 | 1.8 | N/A |
| 2 | | Prostate | African | M | 76 | 2.2 | RT |
| | | | American | | | | |
| 3 | | Prostate | White | M | 79 | 2 | RT |
| | | | | | | | and |
| | | | | | | | ADT |
| 4 | 11 | Prostate | White | M | 75 | 2.3 | ADT |
| 5 | | Prostate | African | M | 71 | — | N/A |
| | | | American | | | | |
| 6 | | Prostate | White | M | 82 | 1.9 | N/A |
| 7 | 16.5 | Prostate | White | M | 68 | — | N/A |
| 8 | | Prostate | White | M | 68 | 1.7 | RT |
| 9 | | Prostate | White | M | 65 | 1.7 | RT |
| | | | | | | | and |
| | | | | | | | ADT |
| 10 | 33 | Prostate | White | M | 91 | 1.8 | N/A |
| 11 | | Multiple | White | M | 57 | 2.4 | RT, |
| | | Myeloma | | | | | RVD, |
| | | | | | | | and |
| | | | | | | | CT |
| 12 | | MPNST | Asian | M | 37 | 1.8 | RT |
| | | | | | | | and |
| | | | | | | | CT |
| 13 | | NSCLC | White | M | 67 | 1.9 | RT |
| | | | | | | | and |
| | | | | | | | CT |
| 14 | | Multiple | White | F | 37 | 1.6 | RVD |
| | | Myeloma | | | | | |
| 15 | 49.5 | NSCLC | African | F | 56 | 1.7 | RT |
| | | | American | | | | and |
| | | | | | | | CT |

Abbreviations:
ADT = Androgen Deprivation Therapy,
BSA = Body Surface Area calculated with Du Bois Formula,
CT = Chemotherapy,
MPNST = Malignant Peripheral Nerve Sheath tumor,
NSCLC = Non-Small Cell Lung Cancer,
RT = Radiation Therapy,
RVD = Lenalidomide, bortezomib and dexamethasone

Study Design: This was a Phase 1 study, with the “3+3” rule used for dose escalation of sodium selenite. Patients were treated in groups of three with each receiving the same dose. Sodium selenite (Biosyn, Germany) was given orally 2 hours prior to scheduled daily radiation therapy treatments for the duration of the radiation therapy course. The initial dose escalation schema was 5.5, 11, 16.5, 33, 49.5, 66, 99, and 121 mg daily. Dose escalation was to proceed as follows: a) if none of the three patients experienced a dose limiting toxicity (DLT), dose escalation to the next dose level would occur, b) if one of three patients treated at that dose level experienced a DLT, that dose level would be expanded to six subjects; if no additional patient in that cohort experienced a DLT, dose escalation to the next dose level would occur, c) if two patients in a cohort experienced a DLT, dose escalation would stop and the prior dose would be considered the maximum tolerated dose (MTD). At that point the MTD was to be expanded to a total of six patients.

Baseline evaluations included EKG, PSA for prostate cancer patients, CBC with differential, CMP, LDH, bone scan (BS), or CT, PET/CT or MRI as clinically indicated to monitor response to therapy. Palliative radiation therapy utilized standard of care palliative dose/fractionation regimens. A summary of radiation therapy parameters and concurrent therapy are summarized in Table 2. On Week 1, Day 1 patients underwent physical exam (PE), laboratory studies as above and EKG. All patients completed a pain inventory, the Brief Pain Inventory, prior to therapy. Sodium selenite was begun 2 hours prior to the scheduled radiation therapy appointment time. Weekly, during radiation therapy, patients had vital signs performed, with assessment of adverse events (AEs), labs and EKG if clinically indicated. The pain inventory was completed again on the last day of radiation therapy. Following completion of therapy, the first follow-up visit was within 2-3 months +/−2 weeks, with subsequent follow-up visits optional until progression of disease at the site of radiation. At these visits patients had a PE, labs and imaging. They also completed another pain inventory.

TABLE 2
Radiation Treatment Parameters

| | | | | Total | |
|---|---|---|---|---|---|
| Patient | Radiation | Dose/Fx† | Number | Dose | Concurrent |
| No. | Field(s) | (cGy) | of Fx† | (cGy) | Therapy |
| 1 | Bilateral Pelvic bones | 800 | 1 | 800 | Abiraterone |
| 2 | Left Shoulder and Left Hip | 400 | 5 | 2000 | Bicalutamide, |
| | | | | | Leuprolide acetate |
| 3 | Bilateral Sacroiliac Joints | 400 | 5 | 2000 | Abiraterone, |
| | | | | | Leuprolide acetate |
| 4 | Bilateral Sacroiliac Joints | 400 | 5 | 2000 | Bicalutamide, |
| | | | | | Leuprolide acetate |
| 5 | Left Pelvis and Proximal | 300 | 10 | 3000 | Leuprolide acetate |
| | Femur | | | | |
| 6 | T3-T6 | 400 | 5 | 2000 | N/A |
| 7 | C7-T4 & Right Humerus | 400 | 5 | 2000 | N/A |
| 8 | L2 | 800 | 3 | 2400 | Enzalutamide |
| 9 | T11 | 2000 | 1 | 2000 | Bicalutamide, |
| | | | | | Leuprolide acetate |
| 10 | L1-L4 | 400 | 5 | 2000 | Enzalutamide, |
| | | | | | Leuprolide acetate |
| 11 | Left Arm, Left and Right | 300 | 10 | 3000 | N/A |
| | Femur, Left Leg | | | | |
| 12 | Right Lung | 500 | 10 | 5000 | Olaratumab |
| 13 | Left Hip | 300 | 10 | 3000 | Pembrolizumab |
| 14 | Right Sacroiliac and | 400 | 5 | 2000 | N/A |
| | Sternum | | | | |
| 15 | Sacrum and Skull/Dura | 400 | 5 | 2000 | N/A |

†Fx = Fraction

Pharmacokinetic Analysis: Pharmacokinetic blood sampling was performed on Day 1 pre-dose, 15 minutes +/−2 minutes, 1 hour +/−5 minutes, 2 hours +/−10 minutes, 4 hours +/−15 minutes, and 24 hours +/−1 hour. During week 2, on Day 1 pre-dose, 1 hour +/−5 minutes, and other optional time points were obtained when feasible.

Pharmacokinetic analysis and model simulations: The PK profile of selenite was characterized using nonlinear mixed effects (NLME) modeling. Using NONMEM software (version 7.4; ICON PLC, Dublin, Ireland), a 1-compartment model with oral absorption was fit to the data. PK parameter estimates obtained from the model included bioavailability (F), clearance (CL/F), and volume of distribution (V/F). These estimates were used to determine the half-life (t1/2) using the relationship t1/2=0.693/kelimination (kelimination=CL/V) and area under the curve (AUC) using the relationship AUC=(F*dose)/CL.

Using the final model and its parameter estimates, a simulation was performed for each dose level (5.5 mg, 11 mg, 16.5 mg, 33 mg, 49.5 mg) assuming a single dose administration. Additionally, using the dose levels 11 mg, 16.5 mg and 33 mg, a dosing regimen (dose, frequency) was proposed to achieve a target selenite concentration of 5-10 μM (395-790 mcg/L). This therapeutic range was determined by concentrations of selenite/selenium that had activity in vitro, as well as PK studies in mice given 2 mg/kg sodium selenite iv, which was a dose that had significant activity in vivo. The PKPDsim package in R was used to perform simulations.

### Results:

Toxicity Profile: Adverse events are summarized in Table 3 by dose level, in terms of classification, type, grade, number of subjects affected and relationship to selenite. At the 5.5, 11 and 16.5 mg dose levels, there were no AEs attributable to the selenite. At the 33 mg dose level the majority of patients had a variety of grade 1 GI toxicities that ranged in attribution from possibly to probable/definitely related. This was the first dose level at which ondansetron and loperamide-Hcl were prescribed prn, and was highly effective in most of the patients. One patient, with a reported low threshold for nausea, had grade 2 nausea and vomiting that was not well controlled with ondansetron and stopped selenite after 4 of 10 planned treatments, with complete resolution of symptoms within 48 hours. Non-GI side effects included one patient with grade 1 fatigue, one patient with grade 1 dizziness, and one patient had grade 2 fatigue, that were possibly related to the selenite treatment. In addition, two patients had grade 1 ECG QTcF prolongations, initially scored as probably related to the selenite. One patient was taken off the study after the first dose of selenite when this occurred given his age of 91 years and relatively frail condition.

Pre and post selenite treatment ECGs were subsequently reviewed by Dr. Philip Sager (Department of Medicine, Stanford University; Executive Committee, Cardiac Safety Research Consortium; personal communication). The observed grade 1 ECG changes were determined to be within the range of expected intra-subject variability, but it was not possible to exclude a potential QTc effect of the selenite. The one patient treated at the 49.5 mg dose level had grade 2 diarrhea, nausea and vomiting that was probably related to selenite, as well as grade 1 fatigue that was possibly related to the treatment. This patient required ondansetron every 8 hours as well as loperamide-Hcl, which improved the symptomatology, but did not completely control it. At that point, although this level of toxicity did not meet the strict definition of a DLT, it was felt that this toxicity profile in this patient population was not acceptable, and the highest dose level that was reasonably well tolerated with ondansetron prn was 33 mg.

TABLE 3
Adverse Events

| | | | | | Number | |
|---|---|---|---|---|---|---|
| Cohort- | | | | | of | |
| Selenite | | | Grade | Number of | Subjects | Relation to |
| Dose (mg) | Classification | Type | Attribution | Occurrences | Affected | Selenite |
| 33 | Blood and | Anemia | 3 | 1 | 1 | Unrelated |
| | Lymphatic | | | | | |
| | System | | | | | |
| | Gastrointestinal | Abdominal | 1 | 3 | 3 | 1-Possible, |
| | | pain | | | | 2-Probably |
| | | Diarrhea | 1 | 5 | 4 | 2-Possible, |
| | | | | | | 3-Probable |
| | | Dysphagia | 1 | 1 | 1 | Unrelated |
| | | Nausea | 1 | 8 | 5 | 7- |
| | | | | | | Probable, |
| | | | | | | 1- |
| | | | | | | Unrelated |
| | | Nausea | 2 | 1 | 1 | Probable/ |
| | | | | | | Definite |
| | | Vomiting | 1 | 6 | 2 | 4-Probable, |
| | | | | | | 2-Unlikely |
| | | Vomiting | 2 | 1 | 1 | Probable |
| | General | Facial Pain | 1 | 1 | 1 | Unrelated |
| | | Fatigue | 1 | 2 | 2 | Possible |
| | | | 2 | 2 | 1 | Unrelated, |
| | | | | | | Possible |
| | | Flu-like | 1 | 2 | 1 | Unrelated |
| | | Symptoms | | | | |
| | Infection | Upper | 1 | 1 | 1 | Unrelated |
| | and | Respiratory | | | | |
| | Infestation | Infection | | | | |
| | Injury | Fall | 2 | 2 | 1 | Unrelated |
| | | (Mechanical) | | | | |
| | Investigations | ECG QTcF | 1 | 2 | 2 | Probable |
| | | Prolonged | | | | |
| | Musculoskeletal | Bone Pain | 1 | 1 | 1 | Unrelated |
| | and | Back Pain | 1 | 1 | 1 | Unrelated |
| | Connective | | | | | |
| | Tissue | | | | | |
| | Nervous | Dizziness | 1 | 1 | 1 | Probable |
| | System | Paresthesia | 1 | 1 | 1 | Unrelated |
| | Respiratory, | Dyspnea | 1 | 1 | 1 | Unrelated |
| | Thoracic | Dyspnea | 2 | 1 | 1 | Unrelated |
| | and | Sore | 1 | 1 | 1 | Unrelated |
| | Mediastinal | Throat | | | | |
| 49.5 | Gastrointestinal | Diarrhea | 2 | 1 | 1 | Possible |
| | | Nausea | 1 | 1 | 1 | Probable |
| | | | 2 | 1 | 1 | Probable |
| | | Vomiting | 1 | 1 | 1 | Probable |
| | | | 2 | 1 | 1 | Probable |
| | General | Fatigue | 1 | 1 | 1 | Possible |
| | Musculoskeletal | Back Pain | 2 | 1 | 1 | Unrelated |
| | and | | | | | |
| | Connective | | | | | |
| | Tissue | | | | | |
| | | Pain in | 2 | 1 | 1 | Unrelated |
| | | Extremity | | | | |

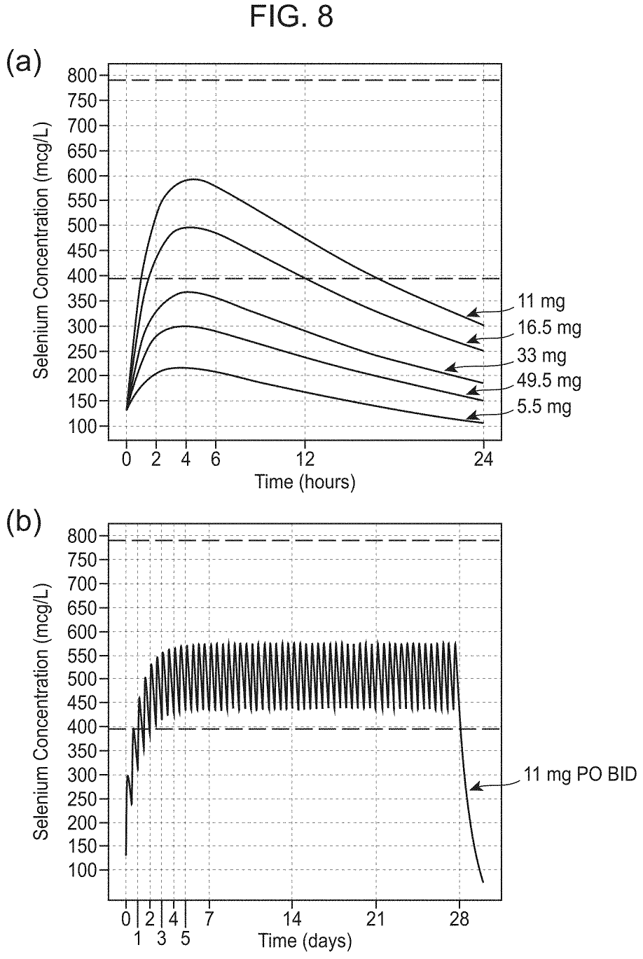

Pharmacokinetic analysis and model simulations: Using the parameter estimates from the population PK model, the half-life was calculated to be approximately 18.5 hours. Table four provides the area under the curve, the maximum concentration (Cmax), and the time to maximum concentration (tmax) for each dose level. FIG. 8(a) provides a simulation using the final parameter estimates from the PK model for each dose level after a single dose administration, which is in alignment with the data that were collected from the study and modeled. This simulation reveals that only the higher dose levels (33 mg and 49.5 mg) reach the desired therapeutic range after a single dose. FIG. 8(b) provides a simulation for a new proposed dosing regimen of 11 mg dosed twice daily to achieve and remain within the desired concentration range.

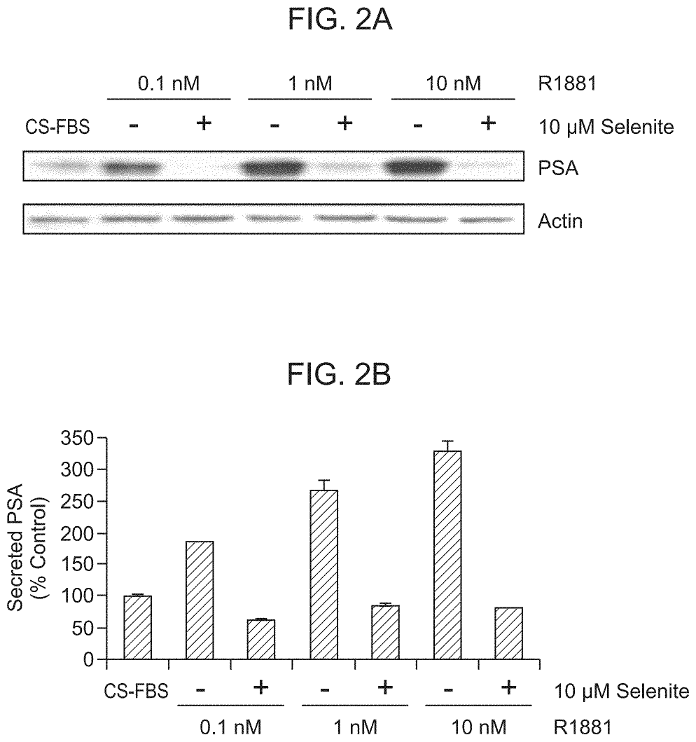

Clinical Responses: It is important to note that patient numbers were small, and the patient population heterogeneous. Nevertheless, data were gathered for PSA in the subset of patients with prostate cancer (Table 5). All patients completed a pain inventory prior to treatment, on the last day of radiation therapy and at their first follow up visit (Table 6). Tumor response in the irradiated field was assessed as well (Table 7). An example of the response observed in a patient with CRPC is shown in FIG. 2. A comparison of pre and post treatment bone scans demonstrates a near complete response in the irradiated left hip in Patient 2.

TABLE 4
PK parameters. Parameters derived
from the final population PK model per dose level.

| Dose | AUC | Cₘₐₓ | tₘₐₓ |
|---|---|---|---|
| (mcg) | (mcg · hour/L) | (mcg/L) | (hour) |
| 5,500 | 3629 | 214.9 | 3.65 |
| 1,100 | 6160 | 300.8 | 4.05 |
| 16,500 | 8277 | 365.6 | 4.15 |
| 33,000 | 13259 | 495.5 | 4.35 |
| 49,500 | 16997 | 593.0 | 4.40 |

TABLE 5
PSA Data

| Cohort- | | PSA | PSA | | | |
|---|---|---|---|---|---|---|
| Selenite | | Value | Value | Time | | |
| dose | Patient | Day 1 | FU | to FU | % | |
| (mg) | Number | (ng/ml) | (ng/ml) | Visit (M) | Change | Change |
| 5.5 | 2 | 39.83 | 8.79 | 2 | −77.9 | ↓ |
| | 3 | 225 | 239 | 2.25 | 6.2 | ↑ |
| 11 | 4 | 131.83 | 35.77 | 3.25 | −72.9 | ↓ |
| | 5 | 40.61 | 19.25 | 4 | −52.6 | ↓ |
| | 6 | 0.98 | 0.32 | 4 | −67.3 | ↓ |
| 16.5 | 7 | 1.74 | 21.85 | 2.25 | 1155.7 | ↑↑ |
| | 8 | 3.3 | 1.34 | 2.75 | −59.4 | ↓ |
| | 9 | 1 | 0.89 | 3.25 | −11 | ↓ |
| 33 | 10 | 196 | 64.46 | 3 | −67.1 | ↓ |

TABLE 6
Average Pain Reduction

| | | | Average change in numerical value from baseline | | | | |
|---|---|---|---|---|---|---|---|
| Dose | | | | Worst Pain Last | Least Pain | | Pain Right |
| Cohort | | Total | Assessment | 24 Hrs Mean ± | Last 24 Hrs | Average Pain | Now |
| (mg) | | (N = 15) | Timepoint | SD | Mean ± SD | Mean ± SD | Mean ± SD |
| 5.5 | mg | 3 | End of RT | −4 ± 1.4 | −1.5 ± 2.1 | −2.5 ± 0.7 | −2 ± 0 |
| | | | First follow-up | −2.5 ± 0.7 | −1.5 ± 1.4 | −1.5 ± 0.7 | −2 ± 0 |
| 11 | mg | 3 | End of RT | −1.7 ± 1.5 | −2.7 ± 3.8 | −2.3 ± 4.0 | 1.7 ± 2.9 |
| | | | First follow-up | −0.7 ± 5.0 | −1.3 ± 4.9 | −0.7 ± 4.6 | 1.7 ± 1.2 |
| 16.5 | mg | 3 | End of RT | −1 ± 1.4 | −1 ± 2.8 | −1.5 ± 3.5 | −0.5 ± 2.1 |
| | | | First follow-up | −0.7 ± 0.6 | −2.3 ± 2.1 | −2 ± 2 | −2.7 ± 3.8 |
| 33 | mg | 5 | End of RT | −1 ± 2 | −0.2 ± 0.5 | −1.2 ± 1.3 | −0.6 ± 1.1 |
| | | | First follow-up | −2 ± 2 | 0 ± 1 | −1 ± 1 | −0.8 ± 1.3 |
| 49.5 | mg | 1 | End of RT | −6 ± 0 | −5 ± 0 | −4.5 ± 0 | −4 ± 0 |

| | | | | |
|---|---|---|---|---|
| First follow-up | −10 ± 0 | −9 ± 0 | −8.5 ± 0 | −8 ± 0 |

Abbreviation:
RT = Radiation Therapy

TABLE 7
Tumor Response in Irradiated Field

| | | | | |
|---|---|---|---|---|
| | | | Imaging | |
| Patient | Tumor | | (months | Modality |
| No. | Response | Field | treatment) | after |

| | | | |
|---|---|---|---|
| 1 | NE | Bone | BS (N/A) |
| 2 | Field 1: CR, Field 2: PR | Bone | BS (2 mo.) |
| 3 | SD | Bone | BS (1 mo.) |
| 4 | CR | Bone | BS (1 mo.) |
| 5 | SD ( ↓ Intensity of uptake) | Bone | BS (3 mo.) |
| 6 | PR (Change from diffuse to patch involvement | Bone | BS (5 mo.) |
| | with ↓ intensity of uptake) | | |
| 7 | SD (↓ intensity of uptake) | Bone | BS (1 mo.) |
| 8 | SD | Bone | MRI (2 mo.) |
| 9 | SD | Bone | CT and MRI (3 mo.) |
| 10 | Field 1: PD, Field 2: SD | Bone | CT (4 mo.) |
| 11 | PR (almost complete metabolic resolution) | Bone | PET-CT (6 mo.) |
| 12 | SD | Lymph Node | CT (1 mo.) |
| 13 | PD | Bone | CT, MRI and PET-CT |
| | | | (1 mo.) |
| 14 | NE (no FU scan because of clinical progression) | Bone | PET-CT (N/A) |
| 15 | SD | Bone | BS (2 mo.) |

```
NE: Not Evaluated; no follow-up imaging
For CT or MRI Imaging: PR ≥ 30% ↓ sum of diameters
                        PD ≥ 20% ↑ sum of diameters
For Bone Scan: CR resolution of uptake
            PR decrease in extent of involvement (# and size of lesion ≥ 50% observed)
            SD same extent of involvement (# and size of lesions)
```

Table 5 shows PSA values on Day 1 prior to the initiation of selenite and radiation therapy and the PSA value at the time of the first follow up, which ranged from 2-4 months following the completion of radiation therapy. Seven out of nine patients had a decrease in PSA with the magnitude of change ranging from an 11% to a 77.9% decrease. There was no evidence of a selenite dose response relationship, but patient numbers were too small and the patients too heterogeneous to draw a definitive conclusion about the presence or absence of a dose response relationship. Of note, patients were maintained on whatever ADT they were on before study entry, as discontinuation of ADT could have been even more of a confounding variable. Interestingly, two of the three patients that were not receiving ADT had an increase in PSA.

The pain inventory captured four categories of pain as follows: worst pain in last 24 hours, least pain in last 24 ours, average pain and pain right now. Pain was scored using a 10 point scale, on which 0 was no pain and 10 was severe pain. Table 6 shows the average change in numerical value compared to baseline for two time points, baseline compared to the last day of radiation therapy, and baseline compared to the first follow up visit as a function of dose level. A change of at least one point was felt to be clinically meaningful. As can be seen, there was a generalized improvement in pain in all four categories at both time points, with a suggestion of a dose response relationship with some increase in pain (decreased change from baseline) in the lowest dose groups of 5.5 and 11 mg, but sustained or improved pain in the higher dose groups. These results are potentially confounded by a variety of factors including extent of disease, systemic therapy and pain medicine usage.

Lastly, tumor responses in the irradiated field are summarized in Table 7. All but one patient received palliative radiation therapy to symptomatic bone metastases. Quantitation of response in bone is difficult and imprecise. Furthermore, patients were imaged as per standard of care, using a variety of imaging modalities, including bone scan, which is not applicable to RECIST scoring. Acknowledging these limitations, of the evaluable patients (n=13), eight patients had stable disease (SD) within the irradiated field (includes patient 10 with two sites of disease irradiated, with site dependent SD and progressive disease (PD) in the two sites respectively. Another patient also had PD. The remaining patients had significant improvement with complete resolution of bone scan abnormalities in two irradiated fields (patients two and four). Patient 2 also had a greater than 50% decrease in the number and size of lesions on bone scan, and patient 11 had almost complete metabolic resolution on PET CT. There was no apparent dose response relationship.

Given the promising results with inorganic sodium selenite in preclinical tumor models and some early clinical trials, there is increasing interest in using selenite as a cytotoxic agent, and/or as a sensitizer. For example, in a study of newly diagnosed patients with non-Hodgkin's lymphoma treated with standard chemotherapy with or without adjuvant sodium selenite (0.2 mg/kg/day for 30 days), the patients receiving selenite had down-regulated levels of Bcl-2 and improved clinical outcomes. In another study of selenite (0.2 mg/kg/day for seven days) in combination with chemotherapy, addition of selenite resulted in a significant increase in the percentage of apoptotic lymphoma cells and clinical response compared to patients treated with chemotherapy alone. Sodium selenite has also been studied in a variety of other tumor types, including colon cancer, and head and neck cancer. In addition, patients with multiple tumor types were enrolled in a Phase one trial: the SECAR study, in which 34 patients with different resistant tumor types received i.v. sodium selenite daily for 5 consecutive days either for 2 or 4 weeks. The MTD was defined as 10.2 mg/m² with a calculated median plasma half-life of 18.25 hours. The most common side effects were fatigue, nausea and cramps in fingers and legs.

While there have been no clinical trials to date studying sodium selenite as radiosensitizer, a randomized Phase three trial studied the ability of selenium to function as a radioprotector of normal tissues presumed to be secondary to enhanced antioxidant capacity, as organic selenium is used for the synthesis of antioxidant enzymes. This trial compared selenium supplementation (500 ug po on days of radiation therapy and 300 ug on days without radiation) with observation in patients with gynecologic malignancies treated with radiation therapy. Of note, this study utilized relatively low doses of an organic form of selenium, which is metabolized differently from inorganic forms, for purposes of protection rather than sensitization. Corcoran et al, performed a study with sodium selenate (SeO₄⁻, which is not as reactive with thiols as selenite, SeO₃²⁻). In this study, patients with castration-resistant prostate cancer received escalating doses of selenate orally.

Sodium selenite may radiosensitize multiple tumor types. In our preclinical studies, it radiosensitized tumors in vivo and did not sensitize GI epithelium to radiation (in fact it had a slight protective effect), and may significantly increase the therapeutic window for radiation therapy. In the study described here, the safety, tolerability and PK of sodium selenite was studied in 15 patients with advanced/metastatic tumors receiving concurrent sodium selenite with palliative radiation therapy. The 33 mg dose level had acceptable tolerability, with the primary toxicity being grade 1 GI side effects. These side effects were well controlled with ondansetron and loperamide-Hcl prn. It was concluded that 33 mg would be a reasonable dose for future studies when given orally, one time per day, with no oral intake for at least 2 hours prior.

The half-life obtained from the parameter estimates of the PK model is in agreement with what is reported in the literature. The SECAR study reported a median half-life of approximately 18 hours in patients with malignant disease receiving IV sodium selenite as a single agent. The PK model we have developed for this study captures the accumulation of selenite and appropriately characterizes the sparse data and variability in the patient population.

From the simulations for the PK model, it takes approximately 1 day to enter the desired therapeutic range for the 11 mg dose level when given twice daily. While a few dosing regimens are possible, 11 mg was chosen to minimize nausea.

Efforts were made to assess potential efficacy signals. The majority of patients with prostate cancer did exhibit a decrease in PSA following treatment, and the majority of patients on the study had a decrease in pain indices. Lastly, the majority of patients had stabilization of disease within the radiation therapy field(s), with some demonstrating objective evidence of tumor regression. A randomized, well controlled, study is desirable at the 33 mg dose level to determine if selenite results in clinically meaningful improvements in the response to palliative radiation therapy.

What is claimed is:

1. A method of treating an androgen-responsive cancer or an androgen unresponsive/refractory cancer comprising administering to a subject in need thereof a therapeutically effective amount of an inhibitor of a Sp1 transcription factor in combination with androgen deprivation therapy.

2. The method of claim 1, wherein the inhibitor of the transcription factor Sp1 is sodium selenite or a pharmaceutically acceptable salt thereof.

3. The method of claim 2, wherein selenite is administered to the subject prior to radiation therapy.

4. The method of claim 2, wherein the selenite is administered orally at a dose of from 5 to 100 mg per day.

5. The method of claim 2, wherein the selenite is administered orally in a dosing regime using divided doses given 2-3 times daily, with a total daily dose of from 15-100 mg.

6. The method of claim 2, wherein the radiation is palliative radiation.

7. The method of claim 2, wherein the radiation is definitive radiation.

8. The method of claim 1, wherein the cancer is prostate cancer.

9. The method claim 1, wherein the androgen deprivation therapy comprises administering a therapeutically effective amount of an androgen deprivation therapy therapeutic agent selected from the group consisting of a luteinizing hormone-releasing hormone (LHRH) agonist or antagonist, a CYP17 inhibitor, an anti-androgen, and other androgen-suppressing or blocking drugs.

10. The method of claim 1, wherein the androgen deprivation therapy comprises administering a therapeutically effective amount of a drug selected from the group consisting of, but not limited to, abiraterone, ketoconazole, orteronel, galeterone, seviteronel, leuprolide, goserelin, triptorelin, histrelin, buserelin, degarelix, cyproterone acetate, enzalutamide, apalutamide, flutamide, bicalutamide, nilutamide, estrogen, or a combination thereof.

11. The method of claim 1, wherein the androgen-responsive cancer is prostate cancer, breast cancer, salivary gland cancer, bladder cancer, or esophageal cancer.

12. The method of claim 10, wherein the cancer is castrate-resistant prostate cancer.

13. The method of claim 1, wherein the inhibitor of the Sp1 transcription factor is administered to the subject prior to, concurrent with, or subsequent to the androgen deprivation therapy.

14. The method of claim 1, wherein the inhibitor of the Sp1 transcription factor is selected from the group consisting of selenite, Withaferin A, mithramycin, mithramycin SDK (SDK), mithramycin SK (SK), premithrmycin B (PreB), 17-allylamino-17-demethoxygeldanamycin (17-AAG), EC-8042, bisanthracycline WP631, tolfenamic acid (clotam), a tripartite motif 22 (TRIM22) peptide inhibitor, an anti-sense nucleic acid inhibitor, and a small interfering RNA (siRNA).

15. A method of treating cancer, comprising:
administering to a subject in need thereof a therapeutically effective amount of sodium selenite or a pharmaceutically acceptable salt thereof; followed by administering radiation therapy.

16. The method of claim 15, wherein the cancer is a solid cancer, lymphoma or hematopoietic malignancy such as multiple myeloma.

17. The method of claim 16, wherein the cancer is a tumor such as, but not limited to, prostate cancer, small cell lung cancer, multiple myeloma, or malignant peripheral nerve sheath tumor.

18. The method of claim 14, wherein the selenite is administered orally at a dose of from 5 to 100 mg per day.

19. The method of claim 14, wherein the selenite is administered orally in a dosing regime using divided doses given 2-3 times daily, with a total daily dose of from 15-100 mg.

20. The method of claim 14, wherein the radiation is palliative or definitive radiation.
